(19) United States
(12) Patent Application Publication
(10) Pub. No.: US 2025/0255829 A1
(43) Pub. Date: Aug. 14, 2025

(54) Multifunctional Nanoparticles and Uses in Managing Cancer and Cardiovascular Diseases

(71) Applicant: Emory University, Atlanta, GA (US)

(72) Inventors: Lily Yang, Atlanta, GA (US); Lei Zhu, Atlanta, GA (US)

(21) Appl. No.: 18/855,199

(22) PCT Filed: Apr. 7, 2023
(86) PCT No.: PCT/US2023/065539

§ 371 Date: Oct. 8, 2024

Related U.S. Application Data
(60) Provisional application No. 63/328,784, filed on Apr. 8, 2022.

(51) Int. Cl. A61K 9/51; A61K 31/165; A61K 31/475; A61K 47/69

(52) U.S. Cl. CPC A61K 9/5161; A61K 31/165; A61K 31/475; A61K 47/6939

(57) ABSTRACT

This disclosure relates to nanoparticles comprising a cardiovascular agent, a PD-L1 binding agent on the surface, and optionally an anticancer agent. In certain embodiments, the cardiovascular agent is an inhibitor of cholesterol acyltransferase such as avasimibe. In certain embodiments, the nanoparticles comprise a hyaluronic acid core. In certain embodiments, this disclosure relates to methods of treating or preventing cancer and/or atherosclerosis, or other cardiovascular disease by administering an effective amount of nanoparticles disclosed herein to a subject in need thereof.

### CROSS-REFERENCE TO RELATED APPLICATIONS

This application claims the benefit of U.S. Provisional Application No. 63/328,784 filed Apr. 8, 2022. The entirety of this application is hereby incorporated by reference for all purposes.

### STATEMENT REGARDING FEDERALLY SPONSORED RESEARCH OR DEVELOPMENT

This invention was made with government support under CA257861 and CA198913 awarded by the National Institutes of Health. The government has certain rights in the invention.

### INCORPORATION-BY-REFERENCE OF MATERIAL SUBMITTED AS AN XML FILE VIA THE OFFICE ELECTRONIC FILING SYSTEM

The Sequence Listing associated with this application is provided in XML format and is hereby incorporated by reference into the specification. The name of the XML file containing the Sequence Listing is 22095PCT.xml. The XML file is 38 KB, was created on Apr. 6, 2023, and is being submitted electronically via the USPTO patent electronic filing system.

### BACKGROUND

Cancer treatment is normally approached by a combination of chemotherapy, surgery and/or radiation. These approaches often neglect to treat and eliminate small metastatic tumors. PD-L1 is expressed in tumor cells and tumor associated stromal cells, such as fibroblasts and macrophages. Preclinical and clinical studies have investigated the efficacy of monoclonal antibody therapies which act as immune checkpoint blockades in multiple cancer types which have led to the FDA approval of ipilimumab (anti-CTLA-4) for melanoma, nivolumab (anti-PD-1) for melanoma, non-small cell lung cancer (NSCLC) and renal cell carcinoma (RCC), and pembrolizumab (anti-PD-1) for melanoma and NSCLC. Three anti-PD-L1 antibodies have also been approved by the FDA for the treatment of metastatic urothelial carcinoma, NSCLC, melanoma, RCC, and colorectal cancer. However, these therapies are not universally effective, and relapse is common. Thus, there remains a need to develop more efficacious therapeutic approaches.

Cancer and cardiovascular diseases, such as atherosclerosis, are often comorbid and a major cause of death. Patients share risk factors such as smoking, diabetes, and hypertension. Pushparaji et al. report interventional strategies in cancer-induced cardiovascular disease. Current Oncology Reports, 2021, 23: 133. Baik et al. report mechanisms and clinical manifestations of cardiovascular toxicities associated with immune checkpoint inhibitors. Clinical Science. 2021, 135(5):703-24.

Liu et al., report avasimibe exerts anticancer effects on human glioblastoma cells via inducing cell apoptosis and cell cycle arrest. Acta Pharmacologica Sinica, 2021, 42:97-107.

Yang et al. report potentiating the anti-tumor response of CD8+ T cells by modulating cholesterol metabolism. Nature, 2016, 531 (7596):651-5.

Lee et al. report avasimibe encapsulated in human serum albumin blocks cholesterol esterification for selective cancer treatment. ACS nano, 2015, 9(3):2420-32.

Lee et al. report hyaluronic acid nanoparticles for active targeting atherosclerosis. Biomaterials, 2015, 53:341-8.

See also WO 2020/205937, WO 2018/071911, WO 2013/0343996, and WO 2012/031205.

References cited herein are not an admission of prior art.

### SUMMARY

This disclosure relates to nanoparticles comprising a cardiovascular agent, a PD-L1 binding agent on the surface, and optionally an anticancer agent and uses in treating cancer and/or preventing cardiovascular diseases or conditions, such as atherosclerosis. In certain embodiments, the cardiovascular agent is an inhibitor of cholesterol acyltransferase such as avasimibe. In certain embodiments, the nanoparticles comprise hyaluronic acid as a core material. In certain embodiments, the nanoparticles further comprise an amino-terminal fragment (ATF) of urokinase-type plasminogen activator (uPA) linked to the exterior of the nanoparticles.

In certain embodiments, this disclosure relates to methods of treating or preventing cancer, atherosclerosis, or other cardiovascular disease by administering an effective amount of nanoparticles disclosed herein to a subject in need thereof.

In certain embodiments, this disclosure relates to using PD-1Y coated hyaluronic acid nanoparticles comprising avasimibe for methods disclosed herein.

In certain embodiments, this disclosure relates to using ATF and PD-1Y coated hyaluronic acid nanoparticles comprising avasimibe for methods disclosed herein.

In certain embodiments, this disclosure relates to using ATFmmp14 and PD-1Y coated hyaluronic acid nanoparticles comprising avasimibe for methods disclosed herein.

In certain embodiments, methods include treating cancer in a cancer patient population with comorbid atherosclerosis, or at risk of developing atherosclerosis. In certain embodiments, methods include treating cancer in a cancer patient population to overcome resistance to immune checkpoint therapy in human cancers.

In certain embodiments, methods include treating atherosclerosis or other cardiovascular diseases using ATF and PD-1Y coated hyaluronic acid nanoparticles comprising avasimibe or other cardiovascular agent.

In certain embodiments, the PD-L1 binding agent comprises the amino acid sequence of NWNRLSPSNQTEKQAAPHHHHCGAISLHPKAKIEE (SEQ ID NO: 2) or variant thereof. In certain embodiments, the PD-L1 binding agent comprises the amino acid sequence of NWNRLSPSNQTEKQAAP (SEQ ID NO: 8) or variants thereof and CGAISLHPKAKIEE (SEQ ID NO: 9) or variants thereof. In certain embodiments, the variant of SEQ ID NO: 8 has at least 70 or 80 percent sequence identity. In certain embodiments, the variant of SEQ ID NO: 8 has up to 3 amino acid substitutions, deletions, and/or additions. In certain embodiments, the variant of SEQ ID NO: 9 has at least 80 percent sequence identity. In certain embodiments, the variant of SEQ ID NO: 9 has up to 2 amino acid substitutions, deletions, and/or additions.

In certain embodiments, this disclosure relates to pharmaceutical compositions comprising a nanoparticle as reported herein and a pharmaceutically acceptable excipient.

In certain embodiments, this disclosure relates to the use of nanoparticles disclosed herein for treating cancer. In certain embodiments, this disclosure relates to the production of a medicament having nanoparticles disclosed herein for use in treating cancer.

### BRIEF DESCRIPTION OF THE SEVERAL VIEWS OF THE DRAWINGS

FIG. 1A shows a scheme illustrating the production of a multifunctional immuno-therapy nanoparticles having hyaluronic acid substituted with 5 beta-cholanic acid containing avasimibe.

FIG. 1B shows maleimide conjugation of PD-L1 blocking and binding peptide, PD-1Y, to hyaluronic acid nanoparticles loaded with avasimibe via a thiol group on a cysteine.

FIG. 2A shows data on the in vitro characterization of hyaluronic acid nanoparticles (HANP) with avasimibe. PD-1Y linked HANP/avasimibe has a hydrodynamic size of about 220±47 nm.

FIG. 2B shows data using HPLC to detect amounts of avasimibe loading in HANP.

FIG. 2C shows data from cell proliferation assays. HANP/avasimibe had stronger growth inhibition effect on the MC38 mouse colon cancer cells than avasimibe.

FIG. 3A shows that treatment with PD1Y-HANP/avasimibe significantly inhibited tumor growth in a mouse model bearing mouse colon tumors and atherosclerotic plaques. Apoe^{-/-} mice on a high fat diet carrying s.c. mouse colon tumors (a dual disease model) received five i.v. treatments, once every three days, of 10 mg/kg of avasimibe or equivalent PD1Y-HANP/avasimibe, 10 mg/kg of PD1Y peptide equivalent of PD1Y-HANP or PD1Y. Residual tumors were surgically resected 6 days by surgery after the last therapy. Tumors were weighted. PD1Y-HANP/avasimibe treatment had the strongest tumor growth inhibition compared with other treatments.

FIG. 3B Mice after received surgical resection of residual tumors as shown in the study in FIG. 3A were monitored for tumor recurrence and mouse survival. Kaplan-Meier survival curves data on therapeutic responses after treatment with PD1Y-HANP/avasimibe in the dual disease mouse colon cancer model. PD1Y-HANP/avasimibe treated mice had significantly longer survival time compared with all other treatment groups. Four (4) of 5 mice in the PD1Y-HANP/Ava treated group had tumor free survival for over 120 days. There was no tumor growth after re-challenging with 1×10⁵ of MC38 tumor cells in those mice.

FIG. 4A shows the effect of the treatment using avasimibe and PD1Y-HANP/Avasimibe on atherosclerotic plaques in the aorta of the dual disease mice. Apoe−/−mice bearing dual diseases received the treatment. After surgical resection of the primary tumors, tumor recurrence and mouse survival were monitored. Mice with tumor recurrence and reached the endpoint were sacrificed. Aorta tissues of the mice were collected at the time point. Serial frozen tissue sections were cut throughout the tissue blocks and stained with H&E. Size of plaques in all arteries of each of the H&E stained tissue sections were measured and total size of the plaques from eight tissue sections of each mouse was compared with the mean of the total plaque area of the no treatment control (100%). For PD1Y-HANP/Ava group that sacrificed at 120 days, an additional no treatment group that was time equivalent was used as a control for the progression of atherosclerosis.

FIG. 4B shows the effect of PD1Y-HANP/Avasimibe on atherosclerosis at the time of completing the treatment. Apoe−/−mice bearing subcutaneous mouse colon tumors were treated with different reagents every 3-4 days for three treatments. Three days following the last treatment, all mice were sacrificed, and aorta arteries were collected. Plaque volumes were quantified from serial H&E tissue sections. During 12 days of treatment, PD1Y-HANP/Avasimibe treated mice had 56% reduction of the total plaque volume in the dual disease mouse model.

FIG. 5 shows data on mouse survival after resecting primary tumor using PD1Y-HANP/Avasimibe and/or ATFmmp14-HANP/Avasimibe in the dual disease mouse colon cancer model. Apoe−/−mice bearing s.c. MC38 tumors received i.v. treatments of 5 mg/kg of Avasimibe, 5 mg/kg of PD1Y peptide, or 10 mg/mg of SN38 equivalent dose of free drugs or nanoparticle drugs.

FIG. 6 shows data on the detection of the MC38 colon cancer specific IgG antibodies in mouse serum samples. A cell ELISA of MC38 colon cancer cell plated 96-well microplate was used. Mouse serum samples collected from different treatment groups were diluted at 1:100 to 1:500 dilutions. High levels of tumor reactive IgG antibodies were detected at 1:500 dilution. A high level of MC38 specific IgG antibodies in PD1Y-HANP/Avasimibe(Ava) treated mouse serum samples was confirmed by immunofluorescence labeling in tumor tissue sections of MC38 colon tumors, but not in tumor tissue sections of the 4T1 mouse mammary or KC pancreatic cancer (a transgenic mutant Kras driven mouse tumor cell line). Tumor specific reactivity was evaluated in the tissue sections of the MC38 tumors and normal organs of the mice. A high level of tumor specific antibody labeling was detected in the tumors after incubated with the PD1-HANP/Ava treated mouse serum (1:100 dilution), but not with the no treatment control mouse serum or PD1Y-HANP treated mouse serum. PD1Y-HANP/Ava treated mouse serum showed a low level of fluorescence labeling in all normal organs examined suggesting the production of the MC38 colon cancer specific antibody response.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise.

The term “comprising” in reference to a peptide having an amino acid sequence refers a peptide that may contain additional N-terminal (amine end) or C-terminal (carboxylic acid end) amino acids, i.e., the term is intended to include the amino acid sequence within a larger peptide. The term “consisting of” in reference to a peptide having an amino acid sequence refers a peptide having the exact number of amino acids in the sequence and not more or having not more than a range of amino acids specified in the claim. In certain embodiments, the disclosure contemplates that the “N-terminus of a peptide may consist of an amino acid sequence,” which refers to the N-terminus of the peptide having the exact number of amino acids in the sequence and not more or having not more than a range of amino acids specified in the claim however the C-terminus may be connected to additional amino acids, e.g., as part of a larger peptide. Similarly, the disclosure contemplates that the “C-terminus of a peptide may consist of an amino acid sequence,” which refers to the C-terminus of the peptide having the exact number of amino acids in the sequence and not more or having not more than a range of amino acids specified in the claim however the N-terminus may be connected to additional amino acids, e.g., as part of a larger peptide.

The term “nanoparticle” refers to a molecular conglomerate of about between 1 and 1000 nm in diameter. One more molecules or biomolecules linked to the nanoparticle typically refers to covalently attaching the molecules or biomolecules to a polymer based exterior or coating. Within certain embodiment, the compositions and methods disclosed herein may be utilized with a variety of polymer-coated particle such as, e.g., quantum dots (QDs), metal particles, gold, silver, iron, and iron-oxide nanoparticles (IONPs), or polymeric nanoparticles, such as hyaluronic acid nanoparticles. In certain embodiments, for any of the embodiments disclosed herein, it is contemplated that particles larger than 1000 nm in diameter can be used.

“PD-L1” refers to programmed death-ligand 1, also known as CD274 and B7H1. The amino acid sequence of full-length PD-L1 is provided in GenBank as accession number NP_054862.1. PD-L1 is a 290 amino acid protein with extracellular IgV-like and IgC-like domains (amino acids 19-239 of full-length PD-L1), a transmembrane domain and an intracellular domain of approximately 30 amino acids. PD-L1 is constitutively expressed on many cells such as antigen presenting cells (e.g., dendritic cells, macrophages, and B-cells) and on hematopoietic and non-hematopoietic cells (e.g., vascular endothelial cells, pancreatic islets, and sites of immune privilege). PD-L1 is also expressed on a wide variety of tumors, and virally-infected cells and is a component of the immunosuppressive milieu (Ribas 2012, NEJM 366:2517-2519). PD-L1 binds to one of two T-cell co-inhibitors PD-1 and B7-1.

“PD-1” refers to the programmed death-1 protein, a T-cell co-inhibitor, also known as CD279. The amino acid sequence of full-length human PD-1 is provided in GenBank as accession number NP_005009.2 (SEQ ID NO: 1) MQIPQAPWPVVWAVLQLGWRPGWFLDSPDRPWNPPTFSPALLVVTEGDNATFTCSFSN TSESFVLNWYRMSPSNQTDKLAAFPEDRSQPGQDCRFRVTQLPNGRDFHMSVVRARRN DSGTYLCGAISLAPKAQIKESLRAELRVTERRAEVPTAHPSPSPRPAGQFQTLVVGVVGG LLGSLVLLVWVLAVICSRAARGTIGARRTGQPLKEDPSAVPVFSVDYGELDFQWREKTP EPPVPCVPEQTEYATIVFPSGMGTSSPARRGSADGPRSAQPLRPEDGHCSWPL. PD-1 is a member of the CD28/CTLA-4/ICOS family of T-cell co-inhibitors. PD-1 is a 288-amino acid protein with an extracellular N-terminal domain, which is IgV-like, a transmembrane domain and an intracellular domain containing an immunoreceptor tyrosine-based inhibitory (ITIM) motif and an immunoreceptor tyrosine-based switch (ITSM) motif (Chattopadhyay et al 2009, Immunol. Rev.). The PD-1 receptor has two ligands, PD-L1 and PD-L2.

A “specific binding” refers to binding by molecules, such as polynucleotides, antibodies, and other ligands, that are able to bind to or recognize a binding partner (or a limited number of binding partners) to a substantially higher degree than to other, similar biological entities.

As used herein, the term “conjugated” refers to linking molecular entities through covalent bonds (via linking groups), or by other specific binding interactions, such as due to hydrogen bonding and other van der Walls forces. The force to break a covalent bond is high, e.g., about 1500 pN for a carbon-to-carbon bond. The force to break a combination of strong protein interactions is typically a magnitude less, e.g., biotin to streptavidin is about 150 pN. Thus, a skilled artisan would understand that conjugation must be strong enough to bind molecular entities in order to implement the intended results.

A “linking group” refers to any variety of molecular arrangements that can be used to bridge to molecular moieties together. An example formula may be —Rₙ— wherein R is selected individually and independently at each occurrence as: —CRₙRₙ—, —CHRₙ—, —CH—, —C—, —CH₂—, —C(OH)Rₙ, —C(OH)(OH)—, —C(OH)H, —C(Hal)Rₙ—, —C(Hal)(Hal)- , —C(Hal)H—, —C(N₃)Rₙ—, —C(CN)Rₙ—, —C(CN)(CN)—, —C(CN)H—, —C(N₃)(N₃)—, —C(N₃)H—, —O—, —S—, —N—, —NH—, —NRₙ—, —(C═O)—, —(C═NH)—, —(C═S)—, —(C═CH₂)—, which may contain single, double, or triple bonds individually and independently between the R groups. If an R is branched with an Rₙ it may be terminated with a group such as —CH₃, —H, —CH═CH₂, —CCH, —OH, —SH, —NH₂, —N₃, —CN, or -Hal, or two branched Rs may form an aromatic or non-aromatic cyclic structure. It is contemplated that in certain instances, the total Rs or “n” may be less than 100 or 50 or 25 or 10. Examples of linking groups include bridging alkyl groups and alkoxyalkyl groups.

A “subject” is defined to include any living animal or human. The term “non-human animal” includes all vertebrates, e.g., mammals and non-mammals, such as non-human primates, sheep, dog, cow, chickens, amphibians, reptiles, etc. A subject or non-human animal is “treated” if one or more beneficial or desired results, including desirably clinical results, are obtained. For purposes of this disclosure, beneficial or desired clinical results include, but are not limited to, one or more of the following: decreasing one or more symptoms resulting from the disease, increasing the quality of life of those suffering from the disease, decreasing the dose of other medications required to treat the disease, delaying the progression of the disease, and/or prolonging survival of individuals.

Sequence “identity” refers to the number of matching residues (expressed as a percentage) in a sequence alignment between two sequences of the alignment. As used herein, percentage identity of an alignment is calculated using the number of identical positions divided by the greater of the shortest sequence or the number of equivalent positions excluding overhangs wherein internal gaps are counted as an equivalent position. For example, the polypeptides GGGGGG (SEQ ID NO: 10) and GGGGT (SEQ ID NO: 11) have a sequence identity of 4 out of 5 or 80%. For example, the polypeptides GGGPPP (SEQ ID NO: 12) and GGGAPPP (SEQ ID NO: 13) have a sequence identity of 6 out of 7 or 85%.

Percent “similarity” is used to quantify the similarity between two sequences of the alignment. This method is identical to determining the identity except that certain amino acids do not have to be identical to have a match. Amino acids are classified as matches if they are among a group with similar properties according to the following amino acid groups: Aromatic—F Y W; hydrophobic—A V I L; Charged positive: R K H; Charged negative—D E; Polar—S T N Q.

The terms “variant” when used in reference to a polypeptide refer to an amino acid sequence that differs by one or more amino acids from another, usually related polypeptide. The variant may have “conservative” changes, wherein a substituted amino acid has similar structural or chemical properties. One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (in other words, additions), or both. Guidance in determining which and how many amino acid residues may be substituted, inserted, or deleted without abolishing biological activity may be found using computer programs well known in the art, for example, DNAStar™ software. Variants can be tested in functional assays. Certain variants have less than 10%, and preferably less than 5%, and still more preferably less than 2% changes (whether substitutions, deletions, and so on).

In certain embodiments, the disclosure relates to recombinant peptides comprising sequences disclosed herein or variants or fusions thereof wherein the amino terminal end or the carbon terminal end of the amino acid sequence are optionally attached to a heterologous amino acid sequence, label, or reporter molecule. In certain embodiments, the disclosure relates to recombinant peptides comprising sequences disclosed herein or variants or fusions thereof wherein the selected amino acid sequence that are critical for the high affinity binding to the target molecule are optionally attached to a heterologous amino acid sequence, label, or reporter molecule.

The term “fusion” when used in reference to a polypeptide refers to a chimeric protein containing a protein of interest joined to an exogenous protein fragment (the fusion partner). The fusion partner may serve various functions, including enhancement of solubility of the polypeptide of interest, and provide new function of the peptide, as well as providing an “affinity tag” to allow purification of the recombinant fusion polypeptide from a host cell or from a supernatant or from both. If desired, the fusion partner may be removed from the protein of interest after or during purification.

A “label” refers to a detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a “label receptor” refers to incorporation of a heterologous polypeptide in the receptor. A label includes the incorporation of a radiolabeled amino acid, a fluorescent dye, or the covalent attachment of biotinyl moieties to a polypeptide that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionucleotides (such as ³⁵S or ¹³¹I) fluorescent labels (such as fluorescein isothiocyanate (FITC), rhodamine, lanthanide phosphors,, or near infrared dyes), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels are attached by spacer arms of various lengths to reduce potential steric hindrance.

“Radiation therapy” is defined as a cancer treatment that uses high-energy x-rays or other types of radiation to kill cancer cells or keep them from growing. There are two types of radiation therapy. External radiation therapy, which uses a machine outside the body to send radiation to the cancer. Internal radiation therapy uses a radioactive substance sealed in needles, seeds, wires, or catheters that are placed directly into or near the cancer. The way the radiation therapy is administer is directly dependent on the type and stage of the cancer.

“Chemoradiation therapy” is defined as a therapy that combines chemotherapy and radiation therapy to increase the effects of both.

“Cancer” refers any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area. Within the context of certain embodiments, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, HER2 for breast cancer, or others. The cancer to be treated in the context of the present disclosure may be any type of cancer or tumor.

### Treatment of Cancer Patients with Comorbid Atherosclerosis

Immune checkpoint therapy promoted progression and development of unstable atherosclerotic plaques in in cancer patients with comorbid atherosclerosis. In preclinical studies, knocking out PD-1 or PD-L1 gene, or treatment with anti-PD-1 Ab in Ldlr−/−transgenic mice enhanced the development of atherosclerosis and induced unstable plaques. Therefore, cancer patients with clinical atherosclerosis are usually not candidates for anti-immune checkpoint Ab therapy due to the concern of potential side effect. Therefore, the development of improved immunotherapy approaches that have a strong effect on enhancing tumor specific immune response but does not activate immune response in atherosclerotic plaques should allow applications of the improved immunotherapy for cancer patients with atherosclerosis or for decreasing incidence of developing atherosclerosis in the high-risk population. Additionally, targeted delivery of immune checkpoint inhibitors into tumors can reduce immunotherapy related adverse effects (irAEs) in normal organs since nanoparticles are unable to pass through the endothelial cell layer of normal vessels.

The objective of this cancer immunotherapy technology is to enable targeted cancer immunotherapy using a bioactive nanoparticle immunotherapy agent with designs to enhance therapeutic response on human cancer while producing therapeutic benefit in cardiovascular diseases and reducing systemic adverse effect. Nanoparticles were engineered with PD-L1 blocking peptides to Hyaluronic acid (HA) nanoparticle encapsulated with avasimibe (Ava), an inhibitor of acyl-Coenzyme A: cholesterol acyltransferase that decreases intracellular cholesterol.

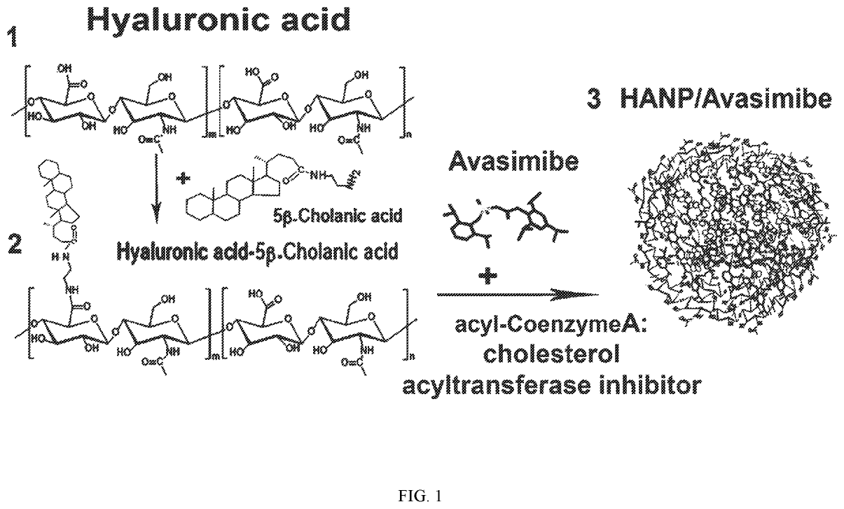

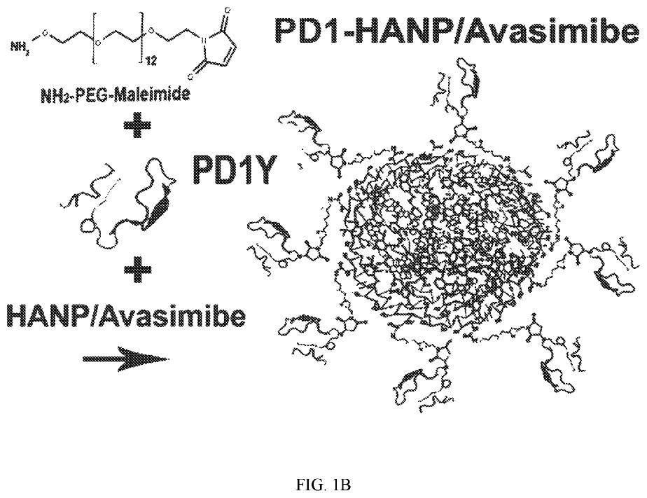

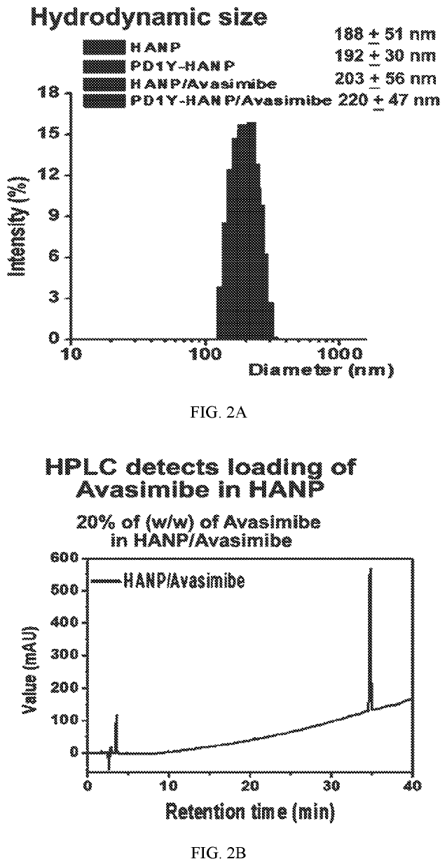

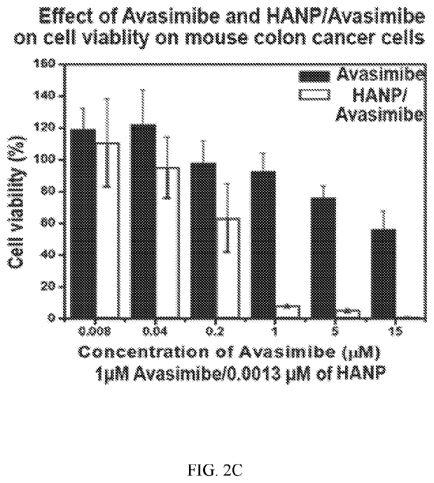

Hydrophobic 5β-cholanic acid was conjugated to HA (HACA) to promote self-assembly and assist in loading of hydrophobic drug molecule avasimibe (Ava). HANP/Ava is produced by self-assembling of HACA and avasimibe using a high-pressure homogenizer. Amount of encapsulated avasimibe could reach to 20% (w/w) in HANP/Ava. PD1Y-HANP/Ava was produced by conjugating chemically synthesized PD1Y peptides, mediated by cysteine of PD1Y and maleimide of a short-PEG (12×) linker (FIG. 1B). A ratio of PD1Y to HANP at 17:1 was used. If a high concentration of PD1Y peptides will be needed for improvement of immune checkpoint inhibition, the amount of PD1Y peptides could be further increased up to 200 peptides per HANP. HANP/Ava treatment inhibited tumor cell proliferation with an IC₅₀ of 280 nM, which was 100-fold higher than avasimibe (IC₅₀=28 μM).

One of the major challenges in immunotherapy of human cancers enriched in tumor stroma is that the stroma barrier prevents efficient intratumoral delivery and distribution of PD-L1 or PD1 antibodies. For example, results of recent clinical trials using antibody therapy to block PD-L1 and PD1 did not show significant therapeutic response in pancreatic cancer patients. The major issues for a poor response in pancreatic cancer are thought to be due to extensive tumor stroma that consists of 50% to 80% of the tumor mass in pancreatic cancer tissue creates physical and biological barriers to block antibody delivery and infiltration of T cells into tumor tissues. Receptor targeted nanoparticle drug carriers disclosed herein have the potential to improve the therapeutic response of immunotherapy through the following mechanisms: 1) targeted delivery of a high level of nanoparticles into tumors that promotes massive infiltration of immune cells, including T cells and antigen presenting cells into tumor center areas to create pro-immune environment to facilitate the activation of immune responses; 2) destroying tumor cells to release tumor specific antigens; band 3) targeted delivery of PD1 like peptide conjugated nanoparticles into tumor that blocks PD-L1 on tumor cells and stromal fibroblasts and macrophages to activate tumor specific T cell responses and to enhance the effect of cytotoxic T cells.

It is contemplated that these nanoparticles can be used as monotherapy or as a combination therapy with chemotherapeutic drugs, radiotherapy, or molecular targeted therapy. Nanoparticles disclosed herein may be produced from hyaluronate (200 kDa) conjugated with a hydrophobic molecule, 5β-cholanic acid, to form HACA nanoparticles. Hyaluronic acid nanoparticles may be loaded with a cancer drug and/or cardiovascular drug produced by conjugation to the out surface of the particle, absorption of the drug by mixing the drug and the nanoparticle, a drug may be produced by self-assembling HACA and the drug using a high-pressure homogenizer, or the cardiovascular drug may be administered separately in avasimibe in combination with HACA nanoparticles coated with PD-1Y.

Preliminary studies used i.p. delivery of unconjugated anti-mouse PD1 antibody in combination with uPAR targeted nanoparticles carrying a chemotherapy drug, cisplatin, demonstrated the feasibility of blocking PD-1/PD-L1 interaction enhanced anti-tumor growth effects of a receptor targeted nanoparticle carrying a chemotherapy drug, cisplatin, in a mouse pancreatic cancer model. PD1-like peptides were designed by selecting the key PD-L1 binding domains of PD1 amino sequences and fusing into a short peptide ligand with 35 (PD1Y) amino acids. These PD1-like peptides have several amino acid modifications to retain domain structures, a short his tag (4) for conjugation and a cysteine (C), for labeling fluorescence dye molecules. In one, a short his tag (4×) was strategically placed in the middle of the PD-1 like peptide (PD1Y) to create a 3-D structure for PD-L1 binding. PD1Y peptide containing a poly-histidine tag can be used for conjugation to nitrilotriacetic acid-copper (NTA-Cu) functionalized nanoparticles to ensure the correct orientation of the PD 1 bind domains. In addition, the cysteine in the linker can also be used for conjugation of NH₂-PEG-maleimide to PD1Y peptide, which then enables the conjugation of PD1Y peptides to the surface of HANP/Avasimibe.

PD-1 (Y)
(SEQ ID NO: 2, MW 4111.56)
NWNRLSPSNQTEKQAAPHHHHCGAISLHPKAKIEE

Shown below are sequence comparisons of the fusion peptide, PD-1 Y, and corollary sequences in PD-1 of mouse (m) and human (h) highlighting variant amino acids in bold below in SEQ ID NO: 18,

PD-1 (Y)-
(SEQ ID NO: 2)
NWNRLSPSNQTEKQAAPHHHHCGAISLHPKAKIEE
PD-1 (m)
(SEQ ID NO: 17)
NWNRLSPSNQTEKQAAFC----CGAISLHPKAKIEE
PD-1 (h)
(SEQ ID NO: 18)
NWYRMSPSNQTDKLAAFP----CGAISLAPKAQIKE

In certain embodiments, the disclosure contemplates PD-L1 binding agents comprising the amino acid sequence of NWNRLSPSNQTEKQAAP (SEQ ID NO: 8) or variants thereof and CGAISLHPKAKIEE (SEQ ID NO: 9) or variants thereof.

In certain embodiments, the variant of SEQ ID NO: 8 has at least 70 or 80 percent sequence identity. In certain embodiments, the variant of SEQ ID NO: 8 has up to 3 amino acid substitutions, deletions, and/or additions. In certain embodiments, the variant of SEQ ID NO: 9 has at least 80 percent sequence identity. In certain embodiments, the variant of SEQ ID NO: 9 has up to 2 amino acid substitutions, deletions, and/or additions.

In certain embodiments, this disclosure contemplates incorporation of human variants into the Y fusion peptide such as with one, two, three, four, five, six or seven amino acid substitution(s) such as represented by the following formula.

NWX¹RX²SPSNQTX³KX⁴AAPXXXXCGAISLX⁵PKAX⁶IX⁷E (SEQ ID NO: 19) X¹ is Y or N, X² is M or L, X³ is E or D, X⁴ is L or Q, X⁵ is H or A, X⁶ is Q or K, and X⁷ is E or K.

In certain embodiments, this disclosure contemplates incorporation of human variants providing

(SEQ ID NO: 14)
NWYRMSPSNQTDKLAAPXXXXCGAISLAPKAQIKE,
(SEQ ID NO: 15)
NWYRMSPSNQTDKLAAPXXXCGAISLAPKAQIKE,
(SEQ ID NO: 16)
NWYRMSPSNQTDKLAAPXXXXXCGAISLAPKAQIKE,

wherein each X is individually and independently at each occurrence any wherein each X is individually and independently at each occurrence any amino acid, histidine, serine, alanine, glycine or is any linking group.

In certain embodiments, the molecule that binds or blocks of PD-L1 is a peptide comprising or consisting of NWNRLSPSNQTEKQAAP (SEQ ID NO: 8) or variants thereof and/or CGAISLHPKAKIEE (SEQ ID NO: 9) or variants thereof. In certain embodiments, the molecule that binds or blocks of PD-L1 is a peptide comprising or consisting of NWYRMSPSNQTDKLAA (SEQ ID NO: 23) or variants thereof and/or CGAISLAPKAQIKE (SEQ ID NO: 24) or variants thereof.

In certain embodiments, the variant of SEQ ID NO: 8 has at least 70, 80, 85, 90, or 95% percent sequence identity. In certain embodiments, the variant of SEQ ID NO: 8 has up to 1 or 2 or 3 amino acid substitutions, deletions, and/or additions. In certain embodiments, the variant of SEQ ID NO: 9 has at least 80, 85, 90, or 95% percent sequence identity. In certain embodiments, the variant of SEQ ID NO: 9 has up to 1 or 2 amino acid substitutions, deletions, and/or additions.

In certain embodiments, the variant of SEQ ID NO: 2 has at least 70, 80, 85, 90, or 95% percent sequence identity. In certain embodiments, the variant of SEQ ID NO: 2 has up to 1 or 2 or 3 amino acid substitutions, deletions, and/or additions.

In certain embodiments, the molecule that binds or blocks of PD-L1 is a peptide comprising or consisting of SEQ ID NO: 1 or 2 or variants thereof.

In certain embodiments, the disclosure relates to targeted delivery of nanoparticles into tumors mediated by PD-L1 blocking peptides. In certain embodiments, the disclosure contemplates nanoparticles comprising PD-1 peptides or fragments or PD-1 like peptides with dual binding domains that are fusions with a poly-histidine tag or other heterologous peptides. The peptides may be conjugated to the nanoparticles through affinity interaction with NTA-Cu that is covalently linked to an outer polymer, configured to form ligand-metal complexes with the poly-histidine tag. The peptides may be conjugated to the nanoparticles through its cysteine labeled NH₂-PEG-maleimide, which then conjugated to the surface of carboxyl group of nanoparticles, such as HANPs.

In certain embodiments, the disclosure contemplates targeted delivery of PD-L1 blocking peptides, such as those comprising of consisting of SEQ ID NO: 8 or variants and/or SEQ ID NO: 9 or variants, such as SEQ ID NO: 2, or variants, into tumors to reduce potential systemic effects of blocking the PD-1 and PD-L1 interaction on the regulation of normal immune responses.

In certain embodiments, the disclosure contemplates methods of treating cancer comprising administering and effective amount of a peptide disclosed herein or a nanoparticle comprising a surface peptide disclosed herein such as those comprising of consisting of SEQ ID NO: 8 or variants and/or SEQ ID NO: 9 or variants, such as SEQ ID NO: 2, or variants in combination with a nanoparticle comprising an amino-terminal fragment (ATF) of uPA to enhance targeting and receptor-mediated internalization of nanoparticle/drug. In certain embodiments, the components are on a single particle or are contained on two particles, e.g., a first nanoparticle comprising a surface peptide such as those comprising or consisting of SEQ ID NO: 8 or variants and/or SEQ ID NO: 9 or variants, such as SEQ ID NO: 2, or variants and a second nanoparticle comprising an amino-terminal fragment (ATF) of uPA or ATF-MMP14 catalytic domain fusion peptide further comprising a chemotherapy agent. ATF-Matrix MetalloProtease 14 (MMP14) can be used to break tumor stromal barrier so that the PD1-like peptide conjugated nanoparticles can be delivered into tumor center and bind to PDL-1 expressing tumor and stromal cells. In certain embodiments, nanoparticles disclosed herein comprising or consisting of SEQ ID NO: 8 or variants and/or SEQ ID NO: 9 or variants, such as SEQ ID NO: 2, or variants and further comprises the amino-terminal fragment (ATF) of uPA or ATF-MMP14 on the outer surface of the particle.

In certain embodiments, the components are on a single particle or are contained on two particles, e.g., a first nanoparticle comprising a surface peptide comprising or consisting of SEQ ID NO: 8 or variants and/or SEQ ID NO: 9 or variants, such as SEQ ID NO: 2 or variants and a second nanoparticle comprising an amino-terminal fragment (ATF) of uPA further comprising a chemotherapy agent. In certain embodiments, nanoparticles disclosed herein comprise a peptide such as those comprising or consisting of SEQ ID NO: 8 or variants and/or SEQ ID NO: 9 or variants, such as SEQ ID NO: 2 or variants and further comprises the amino-terminal fragment (ATF) of uPA on the outer surface of the particle.

In certain embodiments, variants of NWNRLSPSNQTEKQAAP (SEQ ID NO: 8) are selected from NWNRMSPSNQTEKQAAP (SEQ ID NO: 25), NWNRMSPSNQTDKQAAP (SEQ ID NO: 26), NWNRMSPSNQTDKLAAP (SEQ ID NO: 27), NWNRLSPSNQTEKLAAP (SEQ ID NO: 28), NWNRLSPSNQTDKLAAP (SEQ ID NO: 29), NWYRMSPSNQTEKQAAP (SEQ ID NO: 30), NWYRMSPSNQTDKQAAP (SEQ ID NO: 31), NWYRMSPSNQTDKLAAP (SEQ ID NO: 32), NWYRLSPSNQTEKLAAP (SEQ ID NO: 33), and NWYRLSPSNQTDKLAAP (SEQ ID NO: 34).

In certain embodiments, variants of CGAISLHPKAKIEE (SEQ ID NO: 9) are selected from CGAISLAPKAKIEE (SEQ ID NO: 35), CGAISLAPKAQIEE (SEQ ID NO: 36), CGAISLAPKAQIKE (SEQ ID NO: 24), CGAISLHPKAKIKE (SEQ ID NO: 37), and CGAISLHPKAQIKE (SEQ ID NO: 38).

### Nanoparticles

In certain embodiments, this disclosure relates to nanoparticles comprising a cardiovascular agent, a PD-1 or PD-L1 binding agent on the surface, and optionally an anticancer agent. In certain embodiments, the cardiovascular agent is an inhibitor of cholesterol acyltransferase such as avasimibe. In certain embodiments, the nanoparticles comprise hyaluronic acid polymer core. In certain embodiments, nanoparticles disclosed herein may be conjugated to targeting molecules and may optionally be linked to anti-cancer agents and fluorescent dyes. In certain embodiments, the targeting molecule binds uPAR, EGFR, or HER-2, PMSA, IGF-1R, folate receptor, transferrin receptor, MUC-1, integrin alpha-v beta-3, cell surface nucleolin, CTLA-4, or VEGFR. In certain embodiments, the targeting molecule is an antibody or antibody mimetic, or aptamer of a natural ligand thereof such as the amino-terminal fragment of uPA, EGF, or folic acid.

In certain embodiments, nanoparticles are targeted to urokinase plasminogen activator receptor (uPAR), which is a cell surface receptor that is highly expressed in tumor endothelial, stromal fibroblasts and active macrophages, and cancer cells. Methods for carrying various therapeutic agents in or on the nanoparticles are contemplated.

In certain embodiments, this disclosure relates to nanoparticles comprising a cardiovascular agent, a PD-1 or PD-L1 binding agent on the surface, and optionally an anticancer agent. In certain embodiments, the cardiovascular agent is an inhibitor of cholesterol acyltransferase such as avasimibe and optionally a second targeting molecule and optionally a protease polypeptide. In certain embodiments, the targeting molecule is linked to the nanoparticle and the protease polypeptide is linked to the nanoparticle.

In certain embodiments, the fusion polypeptides disclosed herein may contain one or more linking groups or amino acid spacers between the targeting sequence and a fusion, e.g., protease sequence.

In certain embodiments, nanoparticles disclosed herein may be labeled with a near-infrared (NIR) dye on the thiol-group of cysteine residues. Any peptides disclosed herein may contain a his-tag at the C-terminus or N-terminus. Peptides or other compounds may be conjugated to nanoparticles disclose herein through alternative methods, e.g., 1) affinity binding to a surface polymer with NTA-Cu functional groups or 2) direct conjugation of amine groups of targeting peptides with carboxyl groups on the nanoparticle surface via an amide bond.

In certain embodiments, the nanoparticles are hyaluronic acid nanoparticles. Hyaluronic acid (HA) has affinity for CD44, which is overexpressed in lung, breast, pancreatic, and renal tumors. See Naor et al., CD44: structure, function, and association with the malignant process. Adv. Cancer Res. 1997, 71:241-319. Hyaluronic acid HA is synthesized by cells as a high molecular weight form (HMWHA, 1000 to 8000 kDa) and then degraded into low molecular weight fragments (LMWHA, 20 to 250 kDa) by hyaluronidase II (Hyal 2). LMWHA interacts with CD44 receptor for internalization into endosomes/lysosomes and degradation into oligomeric HA (oligo-HA) by hyaluronidase I (Hyal 1). The level of HA in the blood is low and present as a LMW HA form (100-300 kDa) that is cleared out by sinusoid endothelial cells and macrophages in the liver.

Hyaluronic acid nanoparticles may be producing using LMWHA. Hydrophobic patches contribute to the formation stable secondary structures and prevention of nonspecific interactions with proteins and cells. The anti-fouling and viscoelastic features of HA offer advantages for the production of nanoparticle drug carriers. Interactions of HA with cell receptors in tumor stroma and HA-binding proteins in extracellular matrix are believed to contribute to trafficking of HANPs through tumor stroma.

This disclosure relates to nanoparticles comprising peptide-based antagonist of PD-L1 as a targeting moiety. In certain embodiments, the targeting moiety is a peptide comprising SEQ ID NO: 1 or 2, or variants thereof. When reference is made to a nanoparticle comprising a peptide, it is understood that the peptide is bound to the particle through a polymer coating, either through covalent bonds or other binding interactions, e.g., hydrophobic or hydrophilic binding or chelating interactions. In certain embodiments, a nanoparticle comprising a peptide, and the peptide is bound to the particle mediated by interaction of the short his-tag with NTA-Cu that is conjugated to polymer coating of the nanoparticle.

Within certain embodiment, the compositions and methods disclosed herein may be utilized with a variety of polymer-coated nanoparticles such as, e.g., quantum dots (QDs), metal particles, gold, silver, iron, and iron-oxide nanoparticles (IONPs). IONPs are typically prepared with a mean particle diameter of 3-20 nm or 3-200 nm. IONPs may be prepared by aging a stoichiometric mixture of ferrous and ferric salts in aqueous media under basic conditions. Control over particle size (3-20 nm) and shape is provided by adjusting the pH, ionic strength, and the concentration of the growth solution. The nanoparticles can be functionalized in situ using additives such as organic compounds (e.g., sodium citric) or polymers (e.g., dextran, polyvinyl alcohol). Other metals such as gold, cobalt, nickel, and manganese may be incorporated into the material.

High-temperature decomposition of Fe(CO)₅ in organic solvents is another way to prepare IONPs. Size (3-19 nm) can be varied using alternative temperatures. Flame spray pyrolysis yields a range of magnetite, maghemite and wustite (FeO) particles IONPs. Iron precursor such as Fe(CO)₅ and Fe(NO3)₃ may be used. Flame spray pyrolysis can be used to produce different nanoparticles (TiO₂, ZrO₂, silica, etc.) as well as hybrid particles (e.g. silica-IONPs).

Hydroxyl groups on the nanoparticles provide a place for synthetic attachment of different functional groups. A range of chemistries can be used to stabilize metal nanoparticles, exploiting electrostatic, hydrophobic, chelating, and covalent interactions. Carboxylic acid groups can interact with the surface of nanoparticles by coordination processes. Nanoparticle synthesis in organic solvents is typically conducted in oleic acid. A polymer coating on the nanoparticles is preferred. Polymer attachment to the nanoparticles surface by an initiator fixed to the surface of the nanoparticle sand the polymer is grown from the surface. Alternatively, a functional, pre-formed polymer is grafted onto nanoparticles in situ. Copolymers with hydrophobic groups, carboxylic acid groups, polyethylene glycols, or amine groups are contemplated. Polymers with a hydrophilic block and a hydrophobic block are contemplated. See Yang et al., Clin Cancer Res, 2009 15:4722; Lin et al., Small, 2008, 4(3):334-341; Yu et a., Nanotechnology, 2006, 17:4483-4487; Park et al., J. Mater. Chem., 2009, 19, 6412-6417; Boyer et al. NPG Asia Mater., 2010, 2(1):23-30, Kim et al., Nanotechnology, 2011, 22, 155101; all hereby incorporated by reference in their entirety.

Conjugating molecules or polypeptides to the polymers can be accomplished using a variety of methods. Typically, primary amine containing compounds and proteins may be conjugated to the carboxylic acid groups on the polymer mediated by a coupling reagent such as EDAC. See Yang et al., Small, 2009, 5(2):235-43, hereby incorporated by reference in its entirety. Other coupling methods are contemplated, e.g., poly-histidine sequence may be incorporated by recombinant methods into a polypeptide sequence of the targeting moiety. A poly-histidine chelating agent may be coupled to the polymer surface, e.g., NTA-Ni or NTA-Cu. Mixing the histidine tagged polypeptide sequence attaches it to the polymer surface linked through the chelating agent NTA. The avidin/streptavidin-biotin interactions may be used, e.g., biotin may be coupled to the polymer surface and streptavidin may be expressed as a chimera with the targeting moiety.

In certain embodiments, this disclosure relates to nanoparticles comprising a cardiovascular agent, a PD-1 or PD-L1 binding agent on the surface, and further comprising a recombinant fusion polypeptide comprising a human uPA sequence or segment thereof configured to bind urokinase plasminogen activator receptor (uPAR) and optionally a human metalloprotease sequence or segment thereof configured to catalyze the degradation of an extracellular matrix protein such as, but not limited to, MMP14, MMP15, MMP16, and MMP17, metalloelastase (MMP12), collagenases (MMP1, MMP8, MMP13), gelatinases (MMP2, MMP9), stromelysins, (MMP3, MMP10, MMP11), matrilysin (MMP7, MMP26), enamelysin (MMP20).

In addition to the peptides disclosed herein, the nanoparticles may comprise a second targeting moiety. In certain embodiments the targeting moiety is an amino-terminal fragment (ATF) of uPA, e.g., amino terminal fragment (ATF, 135 aa) of human uPA (17 kDa) SNELHQVPSNCDCLNGGTCVSNKYFSNIHWCNCPKKFGGQHCEIDKSKTCYEGNGHFY RGKASTDTMGRPCLPWNSATVLQQTYHAHRSDALQLGLGKHNYCRNPDNRRRPWCY VQVGLKPLVQECMVHDCADGK (SEQ ID NO: 5); or
ATF, 68 aa of human uPA
SNELHQVPSNCDCLNGGTCVSNKYFSNIHWCNCPKKFGGQHCEIDKSKTCYEGN GHFYRGKASTDTMG (SEQ ID NO: 6), or
ATF, 66 aa of human uPA
SNELHQVPSNCDCLNGGTCVSNKYFSNIHWCNCPKKFGGQHCEIDKSKTCYEGN GHFYRGKASTDC (SEQ ID NO: 3), or
human ATF-MMP14
MSNELHQVPSNCDCLNGGTCVSNKYFSNIHWCNCPKKFGGQHCEIDKSKTC YEGNGHFYRGKASTDGAPIQGLKWQHNEITFCIQNYTPKVGEYATYEAIRKAFRVWES ATPLRFREVPYAYIREGHEKQADIMIFF AEGFHGDSTPFDGEGGFLAHAYFPGPNIGGDT HFDSAEPWTVRNEDLNGNDIFLVAVHELGHALGLEHSSDPSAIMAPFYQWMDTENFV LPDDDRRGIQQLYGGESGFPTKMPPQPRTTSRPSVPDKPKNPTYGPNIHHHHHH (SEQ ID NO: 22). Human ATF-first bold letters are (SEQ ID NO: 7) and second sequence (SEQ ID NO: 42) of bold letters are MMP14 segment.

ATF24 containing a N-terminal polyhistidine is

(SEQ ID NO: 21)
CHHHCLNGGTCVSNKYFSNIHWCNCPKK.

ATF65 is

(SEQ ID NO: 20)
SNELHQVPSNCDCLNGGTCVSNKYFSNIHWCNC
PKKFGGQHCEIDKSKTCYEGNGHFYRGKASTDC

ATF fragments and variants may be produced from E. coli BL21 bacterial expression system using a pET20a plasmid (Invitrogen, Grand Island, NY) containing the appropriate ATF cDNA sequence. ATF peptides with amino acids less than 70 could also be synthesized chemically in vitro. Urokinase plasminogen activator (uPA) is a serine protease that regulates multiple pathways involved in matrix degradation, cell motility, metastasis, and angiogenesis. Interaction of the N-terminal growth factor domain of uPA with its cellular receptor (uPAR) results in the conversion of the plasminogen to a serine protease, which is a central regulator of the activation of other proteases including the matrix metalloproteinases (MMPs). Studies have shown that the uPA/uPAR complex controls the motility of both tumor and endothelial cells. In addition to its role in activation of the process for degradation of extracellular matrix, uPAR also activates α5β1 integrin and ERK signaling through interaction with EGFR and induces cell proliferation. Additionally, the uPA/uPAR complex can bind to the matrix protein, vitronectin, in association with transmembrane integrins, and activate intracellular signaling molecules such as the protein kinases, promoting cell adhesion, proliferation, and migration.

Typically, the catalytic domain of matrix metalloproteinases (MMPs) forming the active site comprises a zinc-binding motif of three histidine residues found in the conserved sequence HEXXHXXGXXH (SEQ ID NO: 39) wherein X is individually at each occurrence any amino acid.

An example is the uPA-ATF68-MMP14_{CD} protein sequence (SEQ ID NO: 40). The bold portion is the AFT68 segment, amino acids 2-69 (SEQ ID NO: 41) and segment after, i.e., amino acids 71-246 including the bold zinc binding domain, is the MMP14_{CD} (SEQ ID NO: 42):

(SEQ ID NO: 40)
MSNELHQVPSNCDCLNGGTCVSNKYFSNIHWCNCPKKFGGQHCEI
DKSKTCYEGNGHFYRGKASTDTMGAPIQGLKWQHNEITFCIQNYT
PKVGEYATYEAIRKAFRVWESATPLRFREVPYAYIREGHEKQADI
MIFFAEGFHGDSTPFDGEGGFLAHAYFPGPNIGGDTHEDSAEPWT
VRNEDLNGNDIFLVAVHELGHALGLEHSSDPSAIMAPFYQWMDTE
NFVLPDDDRRGIQQLYGGESG.

In certain embodiments, the disclosure relates to nanoparticles disclosed herein comprising or consisting of ATF fragments, fusions, variants, or sequences as disclosed herein with greater than 30%, 40%, 50%, 60%, 70%, 80%, 90%, or 95% sequence identity or similarity thereto.

In certain embodiments, the disclosure relates to nanoparticles disclosed herein comprising or consisting of a human uPA ATF fragment sequence of less than 135, 100, 90, 80, 70, 60, 50, 40, 30 amino acids, e.g., fusions, variants, or sequences as disclosed herein with greater than 30%, 40%, 50%, 60%, 70%, 80%, 90%, or 95% sequence identity or similarity thereto.

The uPAR-binding domain of uPA is located to the amino-terminal fragment (ATF) of uPA. Studies have shown that ATF is a potent uPA binding antagonist to its high affinity receptor (uPAR) at the surface of both tumor and endothelial cells. Systemic or local delivery of a non-catalytic amino-terminal fragment (ATF) of uPA (residues 1-135) using an adenoviral vector or conjugated peptides prevents the formation of the uPA/uPAR complex, thus inhibiting tumor growth and angiogenesis. Yang et al., Clin Cancer Res., 2009, 15(14):4722-32, hereby incorporated by reference in its entirety, discuss the preparation of targeted iron oxide nanoparticle using a recombinant peptide containing the amino-terminal fragment of urokinase-type plasminogen activator (uPA) conjugated to magnetic iron oxide nanoparticles amino-terminal fragment conjugated-iron oxide nanoparticle (ATF-IONP). This nanoparticle targets uPA receptor, which is overexpressed in breast cancer tissues.

In certain embodiments, the second targeting moiety is a moiety that binds EGFR or HER-2. The human epidermal growth factor receptor (EGFR) family includes EGFR (HER-1), EGFR-2 (HER-2), EGFR-3 (Her-3) and EGFR 4 (HER-4). The ligands that bind to EGFRs are divided into EGFR-like ligands such as EGF and TGF-α, and the heregulins. These ligands bind to EGFR monomers to promoter receptor dimerization and oligomerization that ultimately results in the activation of the EGFR signaling pathway. This EGFR signaling pathway plays a role in the regulation of cell proliferation, survival, and differentiation.

In certain embodiments, the disclosure relates to particles comprising an inhibitor of cholesterol acyltransferase, a PD-1 or PD-L1 binding agent on the surface of core coated with a polymer, wherein the polymer is optionally conjugated to a targeting moiety, a lysosomally degradable moiety, and/or an anticancer agent such as gemcitabine, doxorubicin, cytosine arabinoside, mitomycin, or any therapeutic agent with that an amine side group.

In certain embodiments, the lysosomally degradable moiety is the polypeptide GFLG (SEQ ID NO: 4) linked to the therapeutic agent. In certain embodiments, the disclosure relates to compositions comprising a polymer conjugated to a targeting moiety, lysosomally degradable moiety, and a therapeutic agent which are described herein. In one example, the lysosomally degradable moiety linked to the therapeutic agent is of the formula:

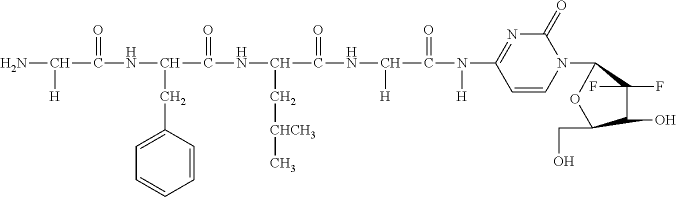

or salts or derivatives thereof optionally substituted with one or more substituents. In certain embodiments, the polymer is an amphiphilic polymer comprising a hydrophobic section further comprising a hydrophobic chemotherapeutic agent.

In certain embodiments, the nanoparticle further comprises a fluorescent dye, e.g., a (3,3-dimethyl-indol-1-ium-1-yl)-N-alkylsulfonate dye or salt thereof such as one of the formula:

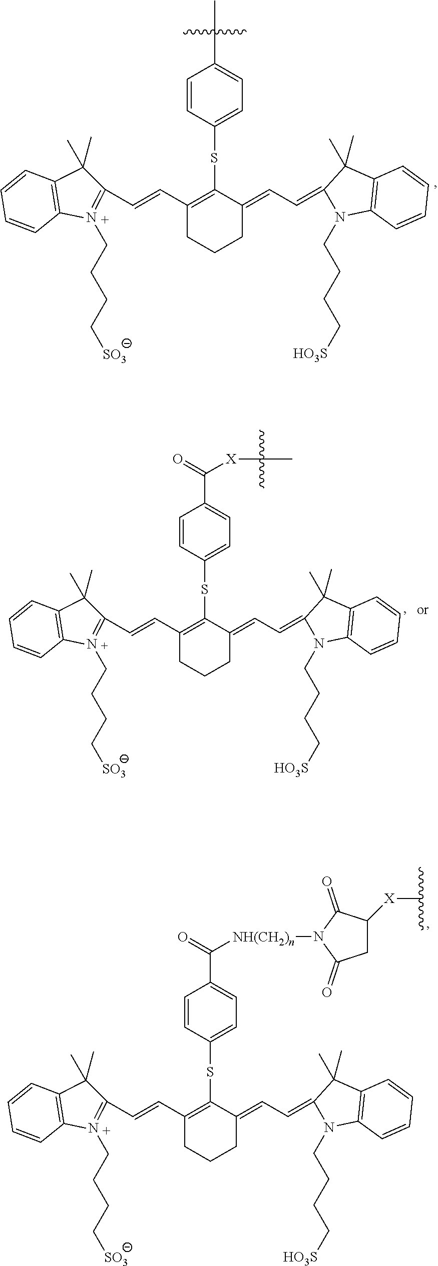

or salts or derivatives thereof optionally substituted with one or more substituents wherein X is S or NH and n is 2 to 22 or n is 4 to 22. In certain embodiments, the dye is conjugated to the free thiol group on cysteine or free amino group of the peptides or proteins.

In certain embodiments, nanoparticles disclosed herein contains a NADPH oxidase (NOX) inhibitor such as setanaxib (GKT831).

### Methods of Use

In certain embodiments, this disclosure relates to methods of treating cancer comprising administering an effective amount of a nanoparticle as disclosed herein to a subject in need thereof optionally in combination with an additional chemotherapy agent or optionally in combination with an additional cardiovascular agent.

In certain embodiments, methods include treating atherosclerosis or other cardiovascular diseases using nanoparticles reported herein comprising avasimibe or other cardiovascular agent. In certain embodiments, this disclosure relates to methods of treating atherosclerosis or other cardiovascular diseases using nanoparticles reported herein comprising administering an effective amount of a nanoparticle reported herein to a subject in need thereof.

In certain embodiments the subject is diagnosed with, exhibiting symptoms of or at risk of atherosclerosis or other cardiovascular disease. In certain embodiments, the subject is diagnosed with clinical symptoms of atherosclerosis such as a plaque rupture or plaque buildup, plaque severe enough to limit or block blood flow, pain can occur in the leg while exercising, erectile dysfunction, heart attack, mini-strokes (transient ischemic attacks), poor wound healing, or stroke. In certain embodiments, the subject is diagnosed with calcific atherosclerosis in their coronary artery and/or aorta, e.g., detected by a CT scan. In certain, embodiments the subject is also diagnosed with cancer, e.g., metastatic colon cancer. In certain embodiments, the additional chemotherapy agent is irinotecan.

In certain embodiments, the subject is diagnosed with cancer and a cardiovascular disease. In certain embodiments, the cardiovascular disease is atherosclerosis, peripheral arterial diseases, coronary artery disease, acute coronary syndrome, stress cardiomyopathy, aortic stenosis, carcinoid heart disease, pericardial effusions, or cardiac masses.

In certain embodiments, the cancer is carcinoma, lymphoma, blastoma, sarcoma, leukemia, non-small cell lung, squamous cell, small-cell lung, peritoneum, hepatocellular, gastrointestinal, pancreatic, glioma, cervical, ovarian, liver, bladder, hepatoma, breast, colon, colorectal, endometrial, uterine, salivary gland, kidney, liver, prostate, vulval, thyroid, hepatic, leukemia and other lymphoproliferative disorders, and various types of head and neck.

In certain embodiments, the anticancer agent for inclusion as a combination therapy with nanoparticles disclosed herein or inclusion as a component in nanoparticles disclosed herein (on the exterior or in the core) is selected from abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelalisib, imatinib, talimogene laherparepvec, ipilimumab, romidepsin, ixabepilone, ixazomib, ruxolitinib, cabazitaxel, palifermin, pembrolizumab, ribociclib, tisagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, zoledronic acid, or combinations thereof.

In certain embodiments, the cardiovascular agent for inclusion as a therapy with nanoparticles disclosed herein or inclusion as a component in nanoparticles disclosed herein (on the exterior or in the core) is selected from apixaban, dabigatran, edoxaban, heparin, rivaroxaban, warfarin, aspirin, clopidogrel, dipyridamole, prasugrel, ticagrelor, benazepril, captopril, enalapril, fosinopril, lisinopril, moexipril, perindopril, quinapril, ramipril, trandolapril, azilsartan, candesartan, eprosartan, irbesartan, losartan, olmesartan, telmisartan, valsartan, sacubitril, acebutolol, atenolol, betaxolol, bisoprolol, hydrochlorothiazide, metoprolol, nadolol, propranolol, sotalol, carvedilol, labetalol, amlodipine, diltiazem, felodipine, nifedipine, nimodipine, nisoldipine, verapamil, atorvastatin, fluvastatin, lovastatin, pitavastatin, pravastatin, rosuvastatin, simvastatin, niacin, ezetimibe, digoxin, isosorbide dinitrate, isosorbide mononitrate, hydralazine, nitroglycerin, minoxidil, or combinations thereof.

In certain embodiments, this disclosure relates to a method of treating cancer comprising administering an effective amount of a nanoparticle comprising an inhibitor of cholesterol acyltransferase, a PD-1 or PD-L1 binding agent on the surface, and optionally an additional anticancer agent as disclosed herein, to a subject in need thereof.

In certain embodiments, this disclosure relates to a method of treating cancer comprising administering an effective amount of a nanoparticle comprising an inhibitor of cholesterol acyltransferase, a PD-1 or PD-L1 binding agent on the surface wherein the PD-L1 binding agent is a peptide comprising SEQ ID NO: 1 or 2, or variants or the PD-1 or PD-L1 binding agent is a corresponding anti-PD-1 or anti-PD-L1 antibody or fragment thereof, to a subject in need thereof

In certain embodiments, the disclosure contemplates a combination chemotherapy comprising the administration of a first agent in combination with a second agent, wherein the first agent is a nanoparticle as disclosed herein and wherein the second agent is a check point inhibitor, such as an anti-PD-L1 antibody, an anti-CTLA-4 antibody such as ipilimumab, or anti-PD-1 antibody such as nivolumab or pembrolizumab.

In certain embodiments, the disclosure contemplates a combination chemotherapy comprising the administration of an inhibitor of a nanoparticle comprising a cholesterol acyltransferase inhibitor optionally in combination with a second anticancer agent, wherein the first agent is a nanoparticle as disclosed herein, wherein the inhibitor of cholesterol acyltransferase is encapsulated by a polymer around the core of the particle.

In certain embodiments, the cancer overexpresses receptors, such as PA-L1 and uPAR, in tumor cells, or tumor stromal fibroblasts and macrophages compared to noncancerous tissue of an organ containing the cancerous tumor. In certain embodiments, the targeting molecule is a peptide inhibitor, or aptamer targeting a protein or glycoprotein expressed on the surface of a cancerous cell. In certain embodiments, the cancer over-expresses uPAR, EGFR, or HER-2, or fragment thereof. In certain embodiments, the cancer is selected from pancreatic cancer, breast cancer, prostate cancer, lung cancer, skin cancer, bladder cancer, brain cancer, colon cancer, rectal cancer, kidney cancer, endometrial cancer, and thyroid cancer.

In certain embodiments, the cancer is selected from carcinoma, lymphoma, blastoma, sarcoma, and leukemia, non-small cell lung, squamous cell, small-cell lung, peritoneum, hepatocellular, gastrointestinal, pancreatic, glioma, cervical, ovarian, liver, bladder, hepatoma, breast, colon, colorectal, endometrial, uterine, salivary gland, kidney, liver, prostate, vulval, thyroid, hepatic, leukemia and other lymphoproliferative disorders, and various types of head and neck. In certain embodiments, the cancer can be primary or metastatic tumors.

In certain embodiments, particles disclosed herein are administered an effective amount to treat a subject diagnosed with cancer or a cancerous tumor. In certain embodiments, the particles disclosed herein are administered in combination with a second anti-cancer agent such as, but not limited to, bevacizumab, gefitinib, erlotinib, temozolomide, docetaxel, cis-platin, 5-fluorouracil, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin, mithramycin, vincristine, vinblastine, vindesine, vinorelbine taxol, taxotere, etoposide, teniposide, amsacrine, topotecan, camptothecin, bortezomib, anagrelide, tamoxifen, toremifene, raloxifene, droloxifene, idoxifene fulvestrant, bicalutamide, flutamide, nilutamide, cyproterone, goserelin, leuprorelin, buserelin, megestrol, anastrozole, letrozole, vorozole, exemestane, finasteride, marimastat, trastuzumab, cetuximab, dasatinib, imatinib, combretastatin, thalidomide, and/or lenalidomide or combinations thereof.

In certain embodiments, the methods reported herein are done in combination with administering an additional chemotherapy agent to the subject. In certain embodiments, the anticancer agent is a checkpoint inhibitor, such as an anti-PD-1, anti-PD-L1 anti-CTLA4 antibody or combinations thereof. In certain embodiments, the anti-CTLA4 antibody is ipilimumab or tremelimumab. In certain embodiments, the anti-PD1 antibody is nivolumab, pembrolizumab, or cemiplimab. In certain embodiments, the anti-PD-L1 antibody is atezolizumab, avelumab, or durvalumab.

In certain embodiments, the subject to be treated has or is diagnosed with a hematological malignancy and has previously received a bone marrow or hematopoietic stem cell transplant, e.g., wherein stem cells are collected from blood or bone marrow.

In certain embodiments, the agent or combination of agents disclosed herein are administered to a subject with a lymphodepleted environment due to prior or concurrent administration of a lymphodepleting agent. In certain embodiments, the of lymphodepleting agent is cyclophosphamide, fludarabine, or combination thereof.

In certain embodiments, the hematological malignancy is selected from leukemia, acute lymphoblastic leukemia (ALL), acute myelogenous leukemia (AML), chronic lymphocytic leukemia (CLL), small lymphocytic lymphoma (SLL), chronic myelogenous leukemia (CML), acute monocytic leukemia (AMOL), myeloproliferative neoplasms (MPNs), and lymphomas, Hodgkin's lymphomas, and non-Hodgkin's lymphomas such as Burkitt lymphoma and other B-cell lymphomas.

In certain embodiments, the methods disclosed herein may be used in combination with radiation and chemoradiation therapy.

In certain embodiments, this disclosure relates to a method for cancer diagnosis comprising administering an effective amount of a nanoparticle disclosed herein to a subject in need thereof and detecting the particle about the area of a cancerous cell or tumor.

Also contemplated are malignancies located in the colon, abdomen, bone, breast, digestive system, liver, pancreas, peritoneum, endocrine glands (adrenal, parathyroid, hypophysis, testicles, ovaries, thymus, thyroid), eye, head and neck, nervous system (central and peripheral), lymphatic system, pelvis, skin, soft tissue, spleen, thorax, childhood acute lymphoblastic leukemia, acute lymphoblastic leukemia, acute lymphocytic leukemia, acute myeloid leukemia, adrenocortical carcinoma, adult (primary) hepatocellular cancer, adult (primary) liver cancer, adult acute lymphocytic leukemia, adult acute myeloid leukemia, adult Hodgkin's disease, adult Hodgkin's lymphoma, adult lymphocytic leukemia, adult non-Hodgkin's lymphoma, adult primary liver cancer, adult soft tissue sarcoma, AIDS-related lymphoma, AIDS-related malignant tumors, anal cancer, astrocytoma, cancer of the biliary tract, cancer of the bladder, bone cancer, brain stem glioma, brain tumors, breast cancer, cancer of the renal pelvis and ureter, primary central nervous system lymphoma, central nervous system lymphoma, cerebellar astrocytoma, brain astrocytoma, cancer of the cervix, childhood (primary) hepatocellular cancer, childhood (primary) liver cancer, childhood acute lymphoblastic leukemia, childhood acute myeloid leukemia, childhood brain stem glioma, childhood cerebellar astrocytoma, childhood brain astrocytoma, childhood extracranial germ cell tumors, childhood Hodgkin's disease, childhood Hodgkin's lymphoma, childhood visual pathway and hypothalamic glioma, childhood lymphoblastic leukemia, childhood medulloblastoma, childhood non-Hodgkin's lymphoma, childhood supratentorial primitive neuroectodermal and pineal tumors, childhood primary liver cancer, childhood rhabdomyosarcoma, childhood soft tissue sarcoma, childhood visual pathway and hypothalamic glioma, chronic lymphocytic leukemia, chronic myeloid leukemia, cancer of the colon, cutaneous T-cell lymphoma, endocrine pancreatic islet cells carcinoma, endometrial cancer, ependymoma, epithelial cancer, cancer of the esophagus, Ewing's sarcoma and related tumors, cancer of the exocrine pancreas, extracranial germ cell tumor, extragonadal germ cell tumor, extrahepatic biliary tract cancer, cancer of the eye, breast cancer in women, Gaucher's disease, cancer of the gallbladder, gastric cancer, gastrointestinal carcinoid tumor, gastrointestinal tumors, germ cell tumors, gestational trophoblastic tumor, head and neck cancer, hepatocellular cancer, Hodgkin's disease, Hodgkin's lymphoma, hypergammaglobulinemia, hypopharyngeal cancer, intestinal cancers, intraocular melanoma, islet cell carcinoma, islet cell pancreatic cancer, Kaposi's sarcoma, cancer of kidney, cancer of the larynx, cancer of the lip and mouth, cancer of the liver, cancer of the lung, lymphoproliferative disorders, macroglobulinemia, breast cancer in men, malignant mesothelioma, malignant thymoma, medulloblastoma, melanoma, mesothelioma, occult primary metastatic squamous neck cancer, primary metastatic squamous neck cancer, metastatic squamous neck cancer, multiple myeloma, multiple myeloma/plasmatic cell neoplasia, myelodysplastic syndrome, myelogenous leukemia, myeloid leukemia, myeloproliferative disorders, paranasal sinus and nasal cavity cancer, nasopharyngeal cancer, neuroblastoma, non-Hodgkin's lymphoma during pregnancy, non-melanoma skin cancer, non-small cell lung cancer, metastatic squamous neck cancer with occult primary, buccopharyngeal cancer, malignant fibrous histiocytoma, malignant fibrous osteosarcoma/histiocytoma of the bone, epithelial ovarian cancer, ovarian germ cell tumor, ovarian low malignant potential tumor, pancreatic cancer, paraproteinemias, purpura, parathyroid cancer, cancer of the penis, phaeochromocytoma, hypophysis tumor, neoplasia of plasmatic cells/multiple myeloma, primary central nervous system lymphoma, primary liver cancer, prostate cancer, rectal cancer, renal cell cancer, cancer of the renal pelvis and ureter, retinoblastoma, rhabdomyosarcoma, cancer of the salivary glands, sarcoidosis, sarcomas, skin cancer, small cell lung cancer, small intestine cancer, soft tissue sarcoma, squamous neck cancer, stomach cancer, pineal and supratentorial primitive neuroectodermal tumors, T-cell lymphoma, testicular cancer, thymoma, thyroid cancer, transitional cell cancer of the renal pelvis and ureter, transitional renal pelvis and ureter cancer, trophoblastic tumors, cell cancer of the renal pelvis and ureter, cancer of the urethra, cancer of the uterus, uterine sarcoma, vaginal cancer, optic pathway and hypothalamic glioma, cancer of the vulva, Waldenstrom's macroglobulinemia, Wilms' tumor and any other hyperproliferative disease, as well as neoplasia, located in the system of a previously mentioned organ.

A “chemotherapy agent,” “chemotherapeutic,” “anti-cancer agent” or the like, refer to molecules that are recognized to aid in the treatment of a cancer. Contemplated examples include the following molecules or derivatives such as temozolomide, carmustine, bevacizumab, procarbazine, lomustine, vincristine, gefitinib, erlotinib, cisplatin, carboplatin, oxaliplatin, 5-fluorouracil, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin, mithramycin, vinblastine, vindesine, vinorelbine, paclitaxel, taxol, docetaxel, etoposide, teniposide, amsacrine, topotecan, camptothecin, bortezomib, anagrelide, tamoxifen, toremifene, raloxifene, droloxifene, idoxifene, fulvestrant, bicalutamide, flutamide, nilutamide, cyproterone, goserelin, leuprorelin, buserelin, megestrol, anastrozole, letrozole, vorozole, exemestane, finasteride, marimastat, trastuzumab, cetuximab, dasatinib, imatinib, combretastatin, thalidomide, azacitidine, azathioprine, capecitabine, chlorambucil, cyclophosphamide, cytarabine, daunorubicin, doxifluridine, epothilone, irinotecan, mechlorethamine, mercaptopurine, mitoxantrone, pemetrexed, tioguanine, valrubicin and/or lenalidomide or combinations thereof such as, cyclophosphamide, methotrexate, 5-fluorouracil (CMF); doxorubicin, cyclophosphamide (AC); mustine, vincristine, procarbazine, prednisolone (MOPP); adriamycin, bleomycin, vinblastine, dacarbazine (ABVD); cyclophosphamide, doxorubicin, vincristine, prednisolone (CHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC).

In certain embodiments, the disclosure relates to methods comprising preoperatively administering cancer targeted nanoparticles conjugated to dyes disclosed herein to a subject, optically imaging a tumor that bind the nanoparticles intra-operatively, and removing tumors targeted with the nanoparticles.

### Pharmaceutical Compositions

In certain embodiments, the disclosure relates to pharmaceutical compositions comprising particles disclosed herein and a pharmaceutically acceptable excipient. Optionally, the pharmaceutical composition further comprises an additional anticancer agent. Optionally, the pharmaceutical composition further comprises an additional cardiovascular agent.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents solvents or vehicles include water, polyethylene glycol, glycerol

Prevention of the action of microorganisms may be controlled by addition of any of various antibacterial and antifungal agents, example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

Pharmaceutical compositions typically comprise an effective amount of particles and a suitable pharmaceutical acceptable carrier. The preparations can be prepared in a manner known per se, which usually involves mixing the particles according to the disclosure with the one or more pharmaceutically acceptable carriers, and, if desired, in combination with other pharmaceutical active compounds and under aseptic conditions. Reference is made to U.S. Pat. Nos. 6,372,778, 6,369,086, 6,369,087 and 6,372,733 and the further references mentioned above, as well as to the standard handbooks, such as the latest edition of Remington's Pharmaceutical Sciences.

The pharmaceutical preparations of the disclosure are preferably in a unit dosage form, and can be suitably packaged, for example in a box, blister, vial, bottle, sachet, ampoule or in any other suitable single-dose or multi-dose holder or container (which can be properly labeled); optionally with one or more leaflets containing product information and/or instructions for use. Generally, such unit dosages will contain between 1 and 1000 mg, and usually between 5 and 500 mg, of the particles of the disclosure e.g., about 10, 25, 50, 100, 200, 300 or 400 mg per unit dosage.

The particles can be administered by a variety of routes including the ocular, transdermal, subcutaneous, intravenous, or intranasal routes, depending mainly on the specific preparation used. The particles will generally be administered in an “effective amount,” by which it is meant any amount of particles that, upon suitable administration, is sufficient to achieve the desired therapeutic or prophylactic effect in the subject to which it is administered. In certain embodiments, it is contemplated that administration is intratumorally once weekly or bi-weekly or by i.v. once weekly or bi-weekly. Usually, depending on the condition to be prevented or treated and the route of administration, such an effective amount will usually be between 0.01 to 1000 mg per kilogram body weight of the subject per treatment, more often between 0.1 and 500 mg, such as between 1 and 250 mg, for example about 5, 10, 20, 50, 100, 150, 200 or 250 mg, per kilogram body weight of the subject per treatment cycle, which can be administered as based on the optimized treatment dose and schedule. The amount(s) to be administered, the route of administration and the further treatment regimen can be determined by the treating clinician, depending on factors such as the age, gender and general condition of the subject and the nature and severity of the disease/symptoms to be treated.

Formulations containing particles described herein can be prepared using a pharmaceutically acceptable carrier composed of materials that are considered safe and effective and can be administered to an individual without causing undesirable biological side effects or unwanted interactions. The carrier is all components present in the pharmaceutical formulation other than the active ingredient or ingredients. As generally used herein “carrier” includes, but is not limited to, diluents, binders, lubricants, disintegrators, fillers, pH modifying agents, preservatives, antioxidants, solubility enhancers, and coating compositions.

In certain embodiments, the composition is a pill, tablet, gel, or in a capsule or the composition is liquid solution such as an aqueous buffer, e.g., a pH of about 6.5, 7.0, or 7.5 or between 6 and 8. In certain embodiments, the pharmaceutically acceptable excipient is selected from a filler, glidant, binder, disintegrant, lubricant, and saccharide.

A “pharmaceutical composition” or “pharmaceutically acceptable” composition, is defined as a therapeutically effective amount of one or more of the compositions described herein, formulated together with one or more pharmaceutically acceptable carriers (additives) and/or diluents. As described in detail, the pharmaceutical compositions of the present disclosure can be specially formulated for administration in liquid form, parenteral administration, for example, by subcutaneous, intramuscular, intravenous, or epidural injection as, for example, a sterile solution or suspension, or sustained-release formulation.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

The compositions of the present disclosure can be given in dosages, generally, at the maximum amount while avoiding or minimizing any potentially detrimental side effects. The compositions can be administered in effective amounts, alone or in a cocktail with other compounds, for example, other compounds that can be used to treat a disease. An effective amount is generally an amount sufficient to inhibit the disease within the subject.

One of skill in the art can determine what an effective amount of the composition is by screening the composition using known methods. The effective amounts may depend, of course, on factors such as the severity of the condition being treated; individual patient parameters including age, physical condition, size, and weight; concurrent treatments; the frequency of treatment; or the mode of administration. These factors are well known to those of ordinary skill in the art and can be addressed with no more than routine experimentation. In some cases, a maximum dose be used, that is, the highest safe dose according to sound medical judgment.

Actual dosage levels of the active ingredients in the pharmaceutical compositions of this disclosure can be varied so as to obtain an amount of the active ingredient that is effective to achieve the desired therapeutic response for a particular patient, composition, and mode of administration, without being toxic to the patient.

The selected dosage level may depend upon a variety of factors including the activity of the particular compound of the present disclosure employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the disclosure employed in the pharmaceutical composition at levels lower than that required to achieve the desired therapeutic effect and then gradually increasing the dosage until the desired effect is achieved.

In some embodiments, a compound or pharmaceutical composition of the disclosure is provided to a subject chronically. Chronic treatments include any form of repeated administration for an extended period of time, such as repeated administrations for one or more months, between a month and a year, one or more years, or longer. In many embodiments, a chronic treatment involves administering a compound or pharmaceutical composition of the disclosure repeatedly over the life of the subject. For example, chronic treatments can involve regular administrations, for example one or more times a day, one or more times a week, or one or more times a month. In general, a suitable dose such as a daily dose of a compound of the disclosure will be that amount of the compound that is the lowest dose effective to produce a therapeutic effect. Such an effective dose will generally depend upon the factors described above.

### Development of an Immune Modulating and Tumor Inhibiting Hyaluronic Acid Nanoparticle Encapsulated with Avasimibe for the Treatment of Cancer Patients with Comorbid Atherosclerosis

Cancer and cardiovascular diseases are the leading causes of death globally. Given the high percentage of cancer patients with co-existing atherosclerosis due to many shared risk factors, the development of cancer therapeutic agents with strong anti-tumor efficacy and therapeutic benefit on atherosclerosis can significantly improve the outcome of cancer therapy. Immune check point inhibition(ICI) therapy using therapeutic antibodies has shown promises in the treatment of several types of human cancers. However, many cancer patients showed a poor response due to low delivery efficiency, lack of effector T cells, an immunosuppressive tumor microenvironment, and intrinsic resistance in solid tumors. Increasing numbers of patients have developed immunotherapy related adverse effects(irAEs). Macrophages and effector T cells drive the progression and destabilization of atherosclerosis. Clinical studies revealed that cancer patients received ICI therapy have 3-fold higher risk of atherosclerosis and 3-fold increases in atherosclerotic plaque progression detected by PET imaging.

To enhance therapeutic efficacy of tumor immunotherapy and decrease irAEs, hyaluronic acid nanoparticles (HANP) were conjugated with PD1 mimetic peptides that target and block PD-L1 function, and avasimibe was encapsulated with the nanoparticles (PD1Y-HANP/Ava). avasimibe is a multifunctional agent that decreases cholesterol accumulation, inhibits tumor growth, and enhances immune response by activating cytotoxic T cells.

Systemic administrations of PD1Y-HANP/Avasimibe led to targeted delivery into tumors and atherosclerotic plaques in a dual colon cancer and atherosclerosis mouse model, established by subcutaneous injection of mouse colon tumor (MC38) cells into Apoe knockout mice on a high fat diet. Five systemic injections of PD1Y-HANP/Avasimibe (10 mg/kg of Avasimibe) resulted in 78% of tumor growth inhibition. Following surgical resection of residual tumors, 80% of PD1Y-HANP/Avasimibe treated mice had disease-free survival of >120 days and were protected from tumor growth after rechallenged with MC38 tumor cells. Histological analysis of all major arteries by H&E staining revealed that the volume of the atherosclerotic plaques decreased by 70% in the mice treated with PD1Y-HANP/Avasimibe compared to non-treated mice. PD1Y-HANP/Avasimibe treatment increased infiltration of CD8+T effector and dendritic cells, and activated cytotoxic T cells, in mouse colon tumors. MC38 tumor specific IgG antibodies were detected in the mouse serum following PD1Y-HANP/Avasimibe treatment. However, the levels of CD8+T cells, dendritic cells, and macrophages were decreased in the atherosclerotic plaques, which could prevent ICI-induced cardiovascular irAEs.

### PD-L1 Targeted PD1Y-HANPs Efficiently Delivered into Tumor and Atherosclerotic Plaques Following Systemic Delivery in the Dual Mouse Colon and Atherosclerosis Model

Apoe^{-/-} (Apo E gene knock-out) mouse is a commonly used mouse model for atherosclerosis. To adequately evaluate targeted delivery and therapeutic efficacy in both tumor and atherosclerotic plaques, a dual disease model was developed by implanting mouse tumor cell lines with a C57BL/6 background, such as mouse colon MC38, into subcutaneous (s.c.) of Apoe^{-/-} mice on a high fat atherogenic diet for 3 to 4 weeks. Apoe^{-/-} mice developed s.c. tumors within 2-3 weeks and extensive atherosclerosis were found in the aortas, especially in the aorta arching. Targeted delivery of PD1Y-HANPs into tumors and atherosclerotic plaques is mediated by targeting PD-L1 (PD1Y) and CD44 (HANP) expressed in both tissues. To determine efficiency of targeted delivery, NIR 830 dye labeled-PD1Y-HANPs were injected i.v. into Apoe^{-/-} mice bearing mouse colon tumors. Optical imaging performed at 48 hrs following injection revealed high levels of the accumulation of the HANPs in tumor tissues in Apoe^{-/-} mice. Histological analysis of H&E stained tissue sections using NIR fluorescence microscopy (Keyence) confirmed HANP signals in the plaques. PD1Y-HANPs were found as clusters in the stromal and tumor areas, with a higher level in necrotic tumor regions. Atherosclerotic plaques on the aorta wall had an intermediate level of NIR signal. Without PD1Y peptide conjugation, NIR 830-HANP/Avasimibe that targets CD44, had low levels of accumulation in tumors and plaques.

### Treatment with PD1Y-HANP/Avasimibe Significantly Inhibited Tumor Growth, Decreased Tumor Recurrence and Increased Mouse Survival in the Dual Disease Mouse Model

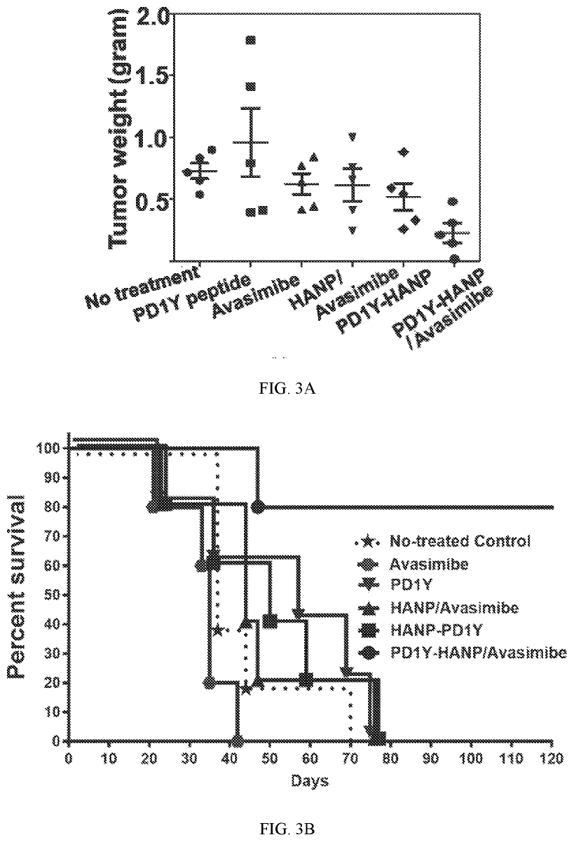

Therapeutic effect of PD1Y-HANP/Avasimibe was examined in the dual disease model. PD1Y-HANP/Avasimibe treatment had the strongest inhibition on tumor growth compared with avasimibe and HANP/Avasimibe treated mouse groups (FIG. 3). Following surgical resection of the residual s.c. tumors, PD1Y-HANP/Avasimibe treated group had significantly reduced tumor recurrence and prolonged mouse survival. Notably, 80% of mice in this group had disease-free survival for more than 120 days and all of the survival mice were protected from tumor growth after re-challenged with MC38 tumor cells. PD1Y-HANP treatment also significantly inhibited tumor growth and delayed tumor recurrence, which led to a modest increase in mouse survival. Unconjugated PD1Y peptide or anti-mouse PD-L1 Ab treatment did not show therapeutic efficacy. There was no systemic toxicity and body weight change following five treatments of PD1Y-HANP/Avasimibe.

### Effect of Targeted Therapy on Atherosclerosis in the Dual Disease Model

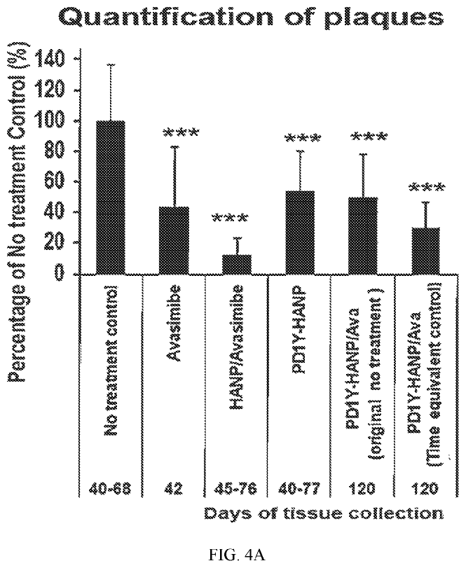

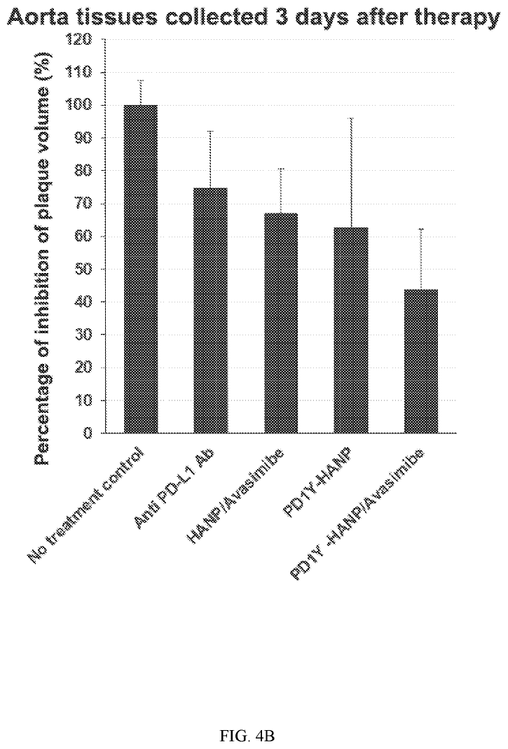

Following treatments and by the end of the survival study, the aorta tissues were collected. Histological analysis of all major arteries by H&E staining revealed that the amount of the plaques was significantly decreased in the mice treated with avasimibe, HANP/Avasimibe and PD1Y-HANP/Avasimibe (FIG. 4A and 4B). HANP/Avasimibe has a stronger inhibitory effect on the plaques than avasimibe.

### uPAR Targeted and Stroma-Penetrating Ligand, ATFmmp14

A uPAR targeted stroma-penetrating ligand (ATFmmp14) was developed containing the amino terminal fragment of uPA fused with the catalytic domain of MMP14. The uPAR targeted and stroma-penetrating ligand, ATFmmp14, has a high delivery efficiency in mouse and human colon tumors, and plaques. Following an i.v. delivery into Apoe−/− mice bearing s.c. mouse colon tumors for 48 hrs, ATFmmp14-HANP and PD1Y-HANPs efficiently delivered into tumor and atherosclerotic plaques, detected by optical imaging and histological analysis. NIR fluorescence microscopy showed that PD1Y-HANPs present as clusters in the stromal and necrotic tumor areas. Notably, ATFmmp14-HANP had a markedly higher level of intratumoral delivery throughout tumor tissues. In mice that received PD1Y-HANP delivery, plaques in the aorta had an intermediate level of NIR signal. However, in mice that received ATFmmp14-HANP had stronger NIR signals in the aorta with plaques than the mice received PD1Y-HANP in both ex vivo imaging and NIR microscopy of tissue sections. ATFmmp14 targets to tumor stromal fibroblasts and macrophages and MMP14 digests extracellular matrixes. A high level of intratumoral delivery in colon PDX tumors were observed in NSG mice. Without PD1Y or ATFmmp14 conjugation, lower levels of HANP/Avasimibe were present in tumors and atherosclerotic plaques. Thus, uPAR targeted ATF or ATFmmp14 conjugated PD1Y-HANP/Avasimibe enhances delivery efficiency in both plaques and tumors in dual disease mouse model or PDX model.

### Comparison of the Therapeutic Effect of ATFmmp14-HANP/SN38 and PD1Y-HANP/Avasimibe Alone or in Combination

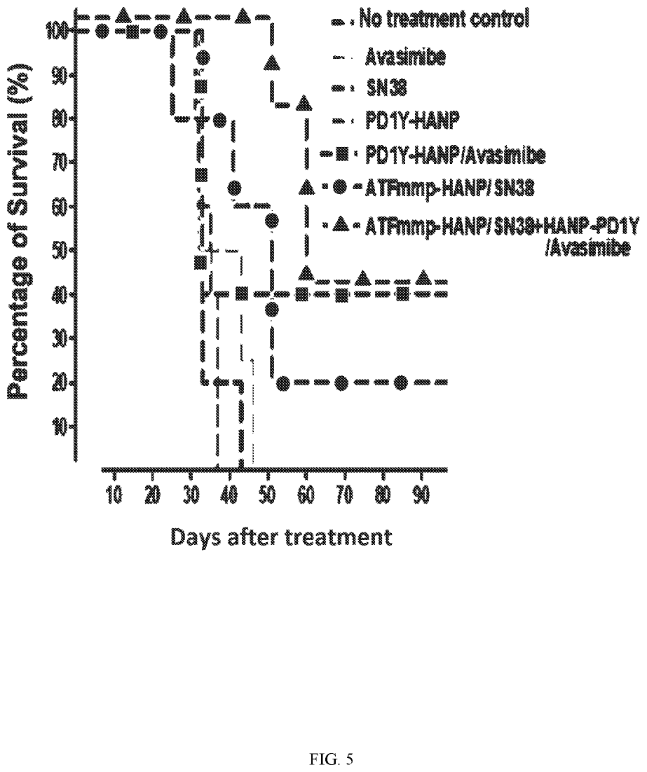

In order to enhance the therapeutic effect a combination of PD-L1 targeted delivery of HANP/Avasimibe was tested with uPAR targeted delivery of SN38 (7-Ethyl-10-hydroxycamptothecin), which is an active metabolite of irinotecan that is commonly used for the treatment of colon cancer. ATFmm14-HANP/SN38 showed therapeutic efficacy in pancreatic and breast PDX models. The effect of ATFmmp14-HANP/SN38 alone or in combination with PD1Y-HANP/Avasimibe at 5 mg/kg of Avasimibe and PD1Y peptide dose/injection was examined in a dual mouse colon cancer and atherosclerosis model. Treatment with PD1Y-HANP/Avasimibe, ATFmmp14-HANP/SN38, or combination of both agents significantly inhibited tumor growth compared with the no-treatment control. ATFmmp14-HANP/SN38 had stronger tumor growth inhibition than the other treatments. However, after surgically resecting the residual tumors, mice treated with the combination of PD1Y-HANP/Avasimibe and ATFmmp 14-HANP/SN38 had reduced tumor recurrence and improved mouse survival (FIG. 5). Significant reductions of the volumes of atherosclerotic plaques in all treatment groups were detected. Avasimibe or SN38 also decreased the plaque volumes.

### Determination of the Differential Effects of PD1Y-HANP/Avasimibe Treatment on Colon Tumors and Atherosclerotic Plaques

Tumors were collected following completing the treatment and aorta tissues were collected when mice reached the end-point. A differential effect of PD1Y-HANP/Avasimibe on immune cells was found with activation and increased infiltration of effector immune cells, such as CD8+, CD68+, CD83+ and CD11C+ cells in tumors, but reduced levels of the above immune effector cells in the plaque tissues. To confirm the effect of the treatment on immune effector cells in tumors and atherosclerotic plaques, the paired tumor and aorta tissues that were collected at the same time point were further analyzed. An additional treatment group that received the combination therapy of PD1Y-HANP/Avasimibe with FOLOX (folinic acid, fluorouracil, and oxaliplatin), the first-line chemotherapy for colon cancer, was included. Results on differential modulation of immune effector cells in tumors and plaques were similar as those observed in the previous study using tissues collected at different time points.

### Induction of Tumor Specific Antibodies in Mice Treated with PD1Y-HANP/Avasimibe

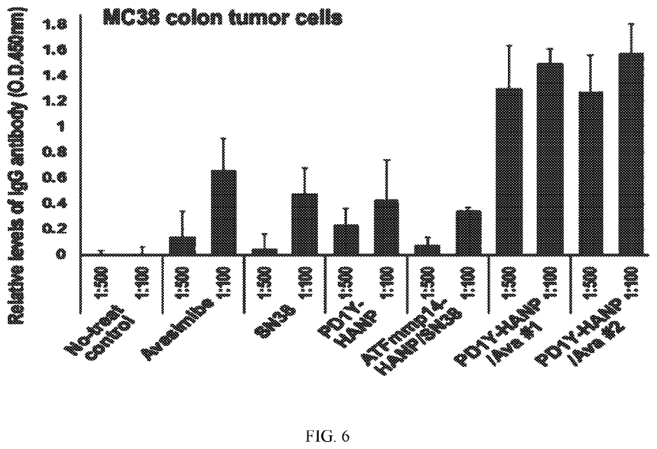

To determine immune response that may contribute to a long-term disease-free survival, mouse serum samples were collected and analyzed for the presence of the MC38 tumor specific antibodies. Cell ELISA assay showed that high levels of IgG antibodies for the MC38 tumor cells were only detected in the serum samples obtained from mice treated with PD1Y-HANP/Avasimibe and survived over 120 days and tumor cell re-challenging (FIG. 6). A strong antibody reactivity of the PD1Y-HANP/Avasimibe treated mouse serum was only detected in tissue sections of the MC38 tumor, but not in mouse mammary and pancreatic tumors. Tumor specificity of the antibodies in the mouse serum obtained from tumor-bearing mice after PD1Y-HANP/Avasimibe.

What is claimed is:

1. A nanoparticle comprising,
an inhibitor of cholesterol acyltransferase,
a PD-L1 binding agent on the surface, and
optionally an additional anticancer agent.

2. The nanoparticle of claim 1 further comprising an amino-terminal fragment of urokinase-type plasminogen activator (uPA) on the surface of the nanoparticle.

3. The nanoparticle of claim 1 wherein the inhibitor of cholesterol acyltransferase is avasimibe.

4. The nanoparticle of claim 1 which is a nanoparticle comprising hyaluronic acid.

5. The nanoparticle of claim 4, wherein avasimibe is in the hyaluronic acid core of the nanoparticle.

6. The nanoparticle of claim 1 wherein the PD-L1 binding agent comprises the amino acid sequence of NWNRLSPSNQTEKQAAP (SEQ ID NO: 8) or variants thereof and CGAISLHPKAKIEE (SEQ ID NO: 9) or variants thereof.

7. The nanoparticle of claim 6, wherein the variant of SEQ ID NO: 8 has at least 70 or 80 percent sequence identity.

8. The nanoparticle of claim 6, wherein the variant of SEQ ID NO: 8 has up to 3 amino acid substitutions, deletions, and/or additions.

9. The nanoparticle of claim 6, wherein the variant of SEQ ID NO: 9 has at least 80 percent sequence identity.

10. The nanoparticle of claim 6, wherein the variant of SEQ ID NO: 9 has up to 2 amino acid substitutions, deletions, and/or additions.

11. The nanoparticle of claim 6, wherein the PD-L1 binding agent comprises the amino acid sequence of NWNRLSPSNQTEKQAAPHHHHCGAISLHPKAKIEE (SEQ ID NO: 2) or variant thereof.

12. A pharmaceutical composition comprising a nanoparticle as in claim 1 and a pharmaceutically acceptable excipient.

13. A method of treating cancer comprising administering an effective amount of a nanoparticle as in claim 1 to a subject in need thereof optionally in combination with administering an additional chemotherapy agent or optionally in combination with an additional cardiovascular agent.

14. The method of claim 13, wherein the subject is diagnosed with cancer and a cardiovascular disease.

15. The method of claim 13, wherein the additional chemotherapy agent is in a nanoparticle comprising hyaluronic acid wherein the additional chemotherapy agent is in the hyaluronic acid core of the nanoparticle.

16. The method of claim 13, wherein the additional chemotherapy agent is 7-ethyl-10-hydroxycamptothecin (SN38) or salt thereof.

17. The method of claim 16, wherein the nanoparticle comprises a recombinant fusion polypeptide comprising a human uPA sequence or segment thereof configured to bind urokinase plasminogen activator receptor (uPAR) and a human metalloprotease sequence or segment thereof configured to catalyze the degradation of an extracellular matrix protein.

18. The method of claim 17, wherein the human metalloprotease sequence is sequence HEXXHXXGXXH (SEQ ID NO: 39) wherein X is individually at each occurrence any amino acid.

19. The method of claim 17, wherein the human uPA sequence or segment thereof configured to bind urokinase plasminogen activator receptor (uPAR) is
(SEQ ID NO: 7)
SNELHQVPSNCDCLNGGTCVSNKYFSNIHWCNC
PKKFGGQHCEIDKSKTCYEGNGHFYRGKASTD.

20. The method of claim 13, wherein the additional chemotherapy agent is folinic acid, fluorouracil, oxaliplatin, or a combination thereof.


SEQUENCE LISTING (42 sequences)

SEQ ID NO: 1 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 35 aa, synthetic construct
NWNRLSPSNQTEKQAAPHHHHCGAISLHPKAKIEE

SEQ ID NO: 3 - protein, 66 aa, synthetic construct
SNELHQVPSNCDCLNGGTCVSNKYFSNIHWCNCPKKFGGQHCEIDKSKTCYEGNGHFYRGKASTDC

SEQ ID NO: 4 - protein, 4 aa, synthetic construct
GFLG

SEQ ID NO: 5 - protein, 135 aa, synthetic construct
SNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCADGK

SEQ ID NO: 6 - protein, 68 aa, synthetic construct
SNELHQVPSNCDCLNGGTCVSNKYFSNIHWCNCPKKFGGQHCEIDKSKTCYEGNGHFYRGKASTDTMG

SEQ ID NO: 7 - protein, 65 aa, synthetic construct
SNELHQVPSNCDCLNGGTCVSNKYFSNIHWCNCPKKFGGQHCEIDKSKTCYEGNGHFYRGKASTD

SEQ ID NO: 8 - protein, 17 aa, synthetic construct
NWNRLSPSNQTEKQAAP

SEQ ID NO: 9 - protein, 14 aa, synthetic construct
CGAISLHPKAKIEE

SEQ ID NO: 10 - protein, 6 aa, synthetic construct
GGGGGG

SEQ ID NO: 11 - protein, 5 aa, synthetic construct
GGGGT

SEQ ID NO: 12 - protein, 6 aa, synthetic construct
GGGPPP

SEQ ID NO: 13 - protein, 7 aa, synthetic construct
GGGAPPP

SEQ ID NO: 14 - protein, 35 aa, synthetic construct
NWYRMSPSNQTDKLAAPXXXXCGAISLAPKAQIKE

SEQ ID NO: 15 - protein, 34 aa, synthetic construct
NWYRMSPSNQTDKLAAPXXXCGAISLAPKAQIKE

SEQ ID NO: 16 - protein, 36 aa, synthetic construct
NWYRMSPSNQTDKLAAPXXXXXCGAISLAPKAQIKE

SEQ ID NO: 17 - protein, 32 aa, synthetic construct
NWNRLSPSNQTEKQAAFCCGAISLHPKAKIEE

SEQ ID NO: 18 - protein, 32 aa, synthetic construct
NWYRMSPSNQTDKLAAFPCGAISLAPKAQIKE

SEQ ID NO: 19 - protein, 35 aa, synthetic construct
NWXRXSPSNQTXKXAAPXXXXCGAISLXPKAXIXE

SEQ ID NO: 20 - protein, 66 aa, synthetic construct; identical to SEQ ID NO: 3

SEQ ID NO: 21 - protein, 28 aa, synthetic construct
CHHHCLNGGTCVSNKYFSNIHWCNCPKK

SEQ ID NO: 22 - protein, 280 aa, synthetic construct
MSNELHQVPSNCDCLNGGTCVSNKYFSNIHWCNCPKKFGGQHCEIDKSKTCYEGNGHFYRGKASTDGAPIQGLKWQHNEITFCIQNYTPKVGEYATYEAIRKAFRVWESATPLRFREVPYAYIREGHEKQADIMIFFAEGFHGDSTPFDGEGGFLAHAYFPGPNIGGDTHFDSAEPWTVRNEDLNGNDIFLVAVHELGHALGLEHSSDPSAIMAPFYQWMDTENFVLPDDDRRGIQQLYGGESGFPTKMPPQPRTTSRPSVPDKPKNPTYGPNIHHHHHH

SEQ ID NO: 23 - protein, 16 aa, synthetic construct
NWYRMSPSNQTDKLAA

SEQ ID NO: 24 - protein, 14 aa, synthetic construct
CGAISLAPKAQIKE

SEQ ID NO: 25 - protein, 17 aa, synthetic construct
NWNRMSPSNQTEKQAAP

SEQ ID NO: 26 - protein, 17 aa, synthetic construct
NWNRMSPSNQTDKQAAP

SEQ ID NO: 27 - protein, 17 aa, synthetic construct
NWNRMSPSNQTDKLAAP

SEQ ID NO: 28 - protein, 17 aa, synthetic construct
NWNRLSPSNQTEKLAAP

SEQ ID NO: 29 - protein, 17 aa, synthetic construct
NWNRLSPSNQTDKLAAP

SEQ ID NO: 30 - protein, 17 aa, synthetic construct
NWYRMSPSNQTEKQAAP

SEQ ID NO: 31 - protein, 17 aa, synthetic construct
NWYRMSPSNQTDKQAAP

SEQ ID NO: 32 - protein, 17 aa, synthetic construct
NWYRMSPSNQTDKLAAP

SEQ ID NO: 33 - protein, 17 aa, synthetic construct
NWYRLSPSNQTEKLAAP

SEQ ID NO: 34 - protein, 17 aa, synthetic construct
NWYRLSPSNQTDKLAAP

SEQ ID NO: 35 - protein, 14 aa, synthetic construct
CGAISLAPKAKIEE

SEQ ID NO: 36 - protein, 14 aa, synthetic construct
CGAISLAPKAQIEE

SEQ ID NO: 37 - protein, 14 aa, synthetic construct
CGAISLHPKAKIKE

SEQ ID NO: 38 - protein, 14 aa, synthetic construct
CGAISLHPKAQIKE

SEQ ID NO: 39 - protein, 11 aa, synthetic construct
HEXXHXXGXXH

SEQ ID NO: 40 - protein, 246 aa, synthetic construct
MSNELHQVPSNCDCLNGGTCVSNKYFSNIHWCNCPKKFGGQHCEIDKSKTCYEGNGHFYRGKASTDTMGAPIQGLKWQHNEITFCIQNYTPKVGEYATYEAIRKAFRVWESATPLRFREVPYAYIREGHEKQADIMIFFAEGFHGDSTPFDGEGGFLAHAYFPGPNIGGDTHFDSAEPWTVRNEDLNGNDIFLVAVHELGHALGLEHSSDPSAIMAPFYQWMDTENFVLPDDDRRGIQQLYGGESG

SEQ ID NO: 41 - protein, 68 aa, synthetic construct; identical to SEQ ID NO: 6

SEQ ID NO: 42 - protein, 11 aa, synthetic construct
HELGHALGLEH